(19) United States
(12) Patent Application Publication
(10) Pub. No.: US 2008/0305485 A1
(43) Pub. Date: Dec. 11, 2008

(54) GENETIC MARKER FOR INCREASED RISK FOR OBESITY-RELATED DISORDERS

(75) Inventors: Steven D. MUNGER, Baltimore, MD (US); Nanette I. Steinle, Millersville, MD (US); Alan R. Shuldiner, Columbia, MD (US); Soren Snitker, Baltimore, MD (US); Cedrick D. Dotson, Baltimore, MD (US); Amanda E.T. Elson, Baltimore, MD (US); Stephen Vigues, Baltimore, MD (US)

Correspondence Address: MORGAN LEWIS & BOCKIUS LLP, 1111 PENNSYLVANIA AVENUE NW, WASHINGTON, DC, 20004, US

(73) Assignee: University of Maryland, Baltimore, MD (US)

(21) Appl. No.: 12/109,137

(22) Filed: Apr. 24, 2008

Related U.S. Application Data
(60) Provisional application No. 60/913,795, filed on Apr. 24, 2007.

(51) Int. Cl. C12Q 1/68; C12N 5/02

(52) U.S. Cl. 435/6; 435/375

(57) ABSTRACT

The present invention relates to methods of determining an increased risk of a subject to acquire a trait of an obesity disorder or an obesity disorder, with the method comprising determining the genetic sequence of at least one taste receptor gene in the subject and reviewing the test genetic sequence(s) for the presence of at least one risk allele associated with at least one taste receptor. The presence of at least one difference in the test genetic sequence(s) and the presence of a risk allele associated with the taste receptor(s) may indicate an increased risk of the subject acquiring a trait of an obesity disorder or an obesity disorder.

### STATEMENT REGARDING FEDERALLY SPONSORED RESEARCH OR DEVELOPMENT

Part of the work performed during development of this invention utilized U.S. Government funds, though NIH Grant Nos. DC005786, DC000054, HL076768, DK072488, DK054261, HL072515, GM074518, DE007309, DC007317 and AG018728, as well as funds through Research Service, Department of Veterans Affairs. The U.S. Government has certain rights in this invention.

### BACKGROUND OF THE INVENTION

1. Field of the Invention

The present invention relates to genetic methods of determining an increased risk of a subject to acquire a trait of an obesity disorder or an obesity disorder.

2. Background of the Invention

Food preference and intake is strongly affected by sweet and bitter taste. For example, individuals who possess enhanced perception of bitter taste avoid certain foods, including specific fruit and vegetables (Drewnowski 1997). Preference for sweet and high-fat food has been reported to decrease with increasing perception of bitter taste (Duffy 2000; Tepper 1997; Tepper 1998). Bitter compound-tasting ability is related to body mass index (BMI), adiposity, and risk factors for CVD (Duffy 2004, Tepper 2002, Goldstein 2005), while the perceived sweetness of foods is inversely correlated with BMI (Bartoshuk 2006). Additionally, bitter taste receptors may play a role in alcohol (Lin 2005) and tobacco addiction (Enoc 2001). In studies designed to detect associations with taste sensitivity and obesity phenotypes, eating behaviors, e.g., dietary restraint and disinhibition, may mask associations with obesity phenotypes and taste sensitivity (Tepper 2002). Much is to yet to be understood regarding the role of taste receptors and nutrient assimilation and metabolism.

Distinct taste receptors are responsible for detection of a number of stimuli, including sweet, umami (glutamate), sour, and bitter compounds. Sweet and umami taste signal the presence of energy rich and essential nutrients and are the main taste attractants in humans, while bitter taste warns of potential toxins (Scott 2005). Therefore, taste is thought to be under strong evolutionary selection. Variations of individual responses, however, to sweet compounds are not yet well characterized. One reason for this poor characterization is modest inter-individual difference in sweet perception, and somewhat weak repeat test reliability detection thresholds for sweeteners (Kim et al. 2004). On the other hand, variation in taste sensitivity to L-glutamate, an umami stimulus, has been demonstrated. Non-tasters have been identified; however the distribution in taste sensitivity among humans appears to be multimodal, suggesting the possibility of the involvement of multiple mechanisms in transmission of umami taste (Lugaz 2002). Using a variety of measures, estimates of the frequency of an individual's ability to detect certain bitter taste have been made in many populations, and it appears that sensitivity to some bitter tastes exhibits a bimodal pattern globally among humans (Tepper 1998). The frequency of individuals insensitive to some bitter tastes among Caucasians is reported to be approximately 28% overall (Kim et al. 2005).

Bitter, sweet and umami tastes are mediated by G-protein-coupled receptors (GPCRs). Bitter taste receptors are encoded by 25-30 TAS2R genes located on chromosomes 12p13, 7q34 and 5p15.31. The ligand specificity of TAS2Rs appears to be quite broad, consistent with their roles in detecting thousands of bitter-tasting compounds (Scott 2005). One of these, TAS2R′38 has been extensively characterized in vitro, in vivo and in human populations, and is responsive to the bitter stimuli phenylthiocarbamide (PTC). Two common haplotypes of TAS2R38 have been shown to influence perception of bitter taste (Kim et al. 2003) and are related to differences in bitter taste sensitivity, to preference for sucrose and sweet tasting foods and beverages, to differences in alcohol consumption, and to modestly lower risk of type 2 diabetes among participants of the British Women's Heart and Health Study (Mennella 2005; Timpson 2005; Wang 2007).

TAS2R5 may be an important regulator of ingestive behavior. The TAS2R5 gene resides in a region of Chromosome 7 that is associated with a quantitative electrophysiological phenotype called tth1, a phenotypic marker of alcohol dependence. Furthermore, a single SNP (single nucleotide polymorphism) located within a linkage disequilibrium block that includes TAS2R5 accounts for this correlation between the receptor and alcohol dependence (Lin 2005). A SNP in another TAS2R receptor located on chromosome 7 has also been associated with alcohol dependence (Hinrichs 2006).

The receptors for sweet and umami taste are encoded by three TAS1R genes located on chromosome 1p36. Heteromeric TAS1R2:TAS1R3 taste receptors respond to sweet-tasting compounds such as sugars, high-potency sweeteners, and some D-amino acids, while TAS1R1:TAS1R3 heteromers comprise an umami taste receptor sensitive to L-amino acids (Scott 2005). Both subunits of the sweet taste receptor bind sugar ligands, though each does so with distinct affinities and with distinct ligand-dependent conformational changes (Nie 2006; Nie 2005).

TAS1Rs and TAS2Rs are expressed in a variety of tissues including brain, adrenal gland, pancreas, small intestine, retina, skeletal muscle, salivary gland and tongue (GEO profiles: www.ncbi.nlm.nig.gov/projects/geo). Of particular interest is the observation that TAS1R and TAS2R receptors, as well as other proteins related to taste transduction, are expressed in rodent and human gastrointestinal mucosa, as well as in STC-1 cells (derived from a small intestine endocrine tumor in a transgenic mouse expressing the rat insulin promoter) and NCI-H716 cells (a human colorectal cell line), where they may be important for modulating responses to ingested nutrients (Dyer 2005; Wu 2002; Bezencon 2007)(Margolskee, 2007; Jang, 2007; Mace, 2007).

TAS1Rs and TAS2Rs expressed in cells of rodent and human gastrointestinal mucosa or in cell lines derived from rodent and human gastrointestinal mucosa respond to TAS1R or TAS2R ligands with increases in intracellular calcium, secretion of incretin hormones such as glucagons-like peptide-1 (GLP-1) and/or changes in expression or localization of glucose transporters (Margolskee, 2007; Jang, 2007; Mace, 2007) such that TAS1Rs and TAS2Rs can mediate nutrient responses, nutrient assimilation, or otherwise respond to the presence of TAS1R or TAS2R ligands in the gut.

The role of genetic variation in specific human taste receptors in the regulation of macronutrient ingestion and the development of obesity and metabolic disorders, however, remains largely unknown. The identification of susceptibility genes for obesity and other metabolic disorders will have significant implications for public health. Knowledge of susceptibility genes could lead to development of screening/diagnostic tests to identify persons at risk of developing obesity or other metabolic disorders. Early ascertainment of heritable obesity risk or metabolic disorder risk would facilitate early implementation of preventive strategies to decrease associated morbidity and mortality. The discovery of polymorphisms in genes that influence nutritional intake may also improve our understanding of the pathogenesis of obesity or other metabolic disorders, lead to earlier diagnosis and treatment, and lead to the development of novel treatments. Finally, testing for susceptibility genes may have prognostic value and may allow physicians to more effectively guide medical therapy.

### SUMMARY OF THE INVENTION

The present invention relates to methods of determining an increased risk of a subject to acquire a trait of an obesity disorder or an obesity disorder, with the method comprising determining the genetic sequence of at least one taste receptor gene in the subject and reviewing the test genetic sequence(s) for the presence of at least one risk allele associated with at least one taste receptor. The presence of at least one difference in the test genetic sequence(s) and the presence of at least one risk allele associated with the taste receptor(s) may indicate an increased risk of the subject acquiring a trait of an obesity disorder or an obesity disorder.

The present invention also relates to a method of diagnosing or testing a subject for an obesity disorder or a trait of an obesity disorder, with the method comprising determining the genetic sequence of at least one taste receptor in the subject. The genetic sequence of the at least one taste receptor in the individual can then be compared to known genetic sequences that are associated with a particular disorder or trait of a disorder. A physician or health care specialist can then use the diagnostic or test results to determine a therapeutic regiment, if necessary.

### BRIEF DESCRIPTION OF THE DRAWINGS

FIG. 1 depicts the schematics of two of the known types of taste receptors, TAS1R and TAS2R.

FIG. 2 depicts the linkage disequilibrium (LD) results of an r² analysis on TAS2R-related SNPs located on chromosome 12.

### DETAILED DESCRIPTION OF THE INVENTION

The present invention relates to methods of determining an increased risk of a subject to acquire a trait of an obesity disorder or an obesity disorder, with the method comprising determining the genetic sequence of at least one taste receptor gene in the subject and reviewing the test genetic sequence(s) for the presence of at least one risk allele associated with at least one taste receptor. The presence of at least one difference in the test genetic sequence(s) and the presence of at least one risk allele associated with the taste receptor(s) may indicate an increased risk of the subject acquiring a trait of an obesity disorder or an obesity disorder.

The present invention is useful for identifying an increased risk in a subject acquiring a trait of an obesity disorder or an obesity disorder. As used herein, an “increased risk” is used to mean that the test subject, who possesses a marker, has an increased chance of developing or acquiring the trait or obesity disorder compared to a subject without the marker.

The increased risk may be relative or absolute and may be expressed qualitatively or quantitatively. For example, an increased risk may be expressed as simply determining the subject's genotype for a given marker and placing the patient in an “increased risk” category, based upon previous population studies. Alternatively, a numerical expression of the subject's increased risk may be determined based upon genotyping analysis. As used herein, examples of expressions of an increased risk include but are not limited to, odds, probability, odds ratio, p-values, attributable risk, relative frequency, positive predictive value, negative predictive value, and relative risk.

For example, the correlation between a marker and a disorder or a trait of a disorder may be measured by an odds ratio (OR) and by the relative risk (RR). If P(R⁺) is the probability of developing the disorder or trait of a disorder for individuals with the marker (R) and P(R⁻) is the probability of developing the disorder or trait of a disorder for individuals without the risk factor, then the relative risk is the ratio of the two probabilities: RR=P(R⁻)/P(R⁻).

In case-control studies, however, direct measures of the relative risk often cannot be obtained because of the sampling design. The odds ratio allows for a good approximation of the relative risk for low-incidence diseases and can be calculated: OR=(F⁺/(1−F⁺))/(F⁻/(1−F⁻)), where F⁺ is the frequency of the exposure to the marker in cases studies and F⁻ is the frequency of the exposure to the risk factor in controls. F⁺ and F⁻ can be calculated using the allelic or haplotype frequencies of the study and may further depend on the underlying genetic model such as, but not limited to dominant models, recessive models and additive models, etc.

The attributable risk (AR) can also be used to express an increased risk. The AR describes the proportion of individuals in a population exhibiting a trait due to a given risk factor, i.e., the marker. AR is important in quantifying the role of a specific marker in disease etiology and in terms of the public health impact of a marker. The public health relevance of the AR measurement lies in estimating the proportion of cases of disease in the population that could be prevented if the marker were absent. AR may be determined as follows: AR=P_{E}(RR−1)/(P_{E}(RR−1)+1), where AR is the risk attributable to a marker allele or a marker haplotype, and P_{E} is the frequency of exposure to an allele or a haplotype within the population at large. RR is the relative risk, which can be approximated with the odds ratio when the marker under study has a relatively low incidence in the general population.

In one embodiment, the increased risk of a patient can be determined from p-values that are derived from association studies. Specifically, associations with single nucleotide polymorphisms (SNSs) can be performed using pedigree-based analysis by regressing the effect of the marker genotype while accounting for residual familial correlations among related individuals. The relatedness among family members may be determined using a measured genotype approach, in which the likelihood of specific genetic models can be estimated given the pedigree structure. For example, the likelihood of a full model which allows for a genotyic-specific means, is compared to a nested model in which genotypic means are restricted to be equal to each other. Parameter estimates are obtained by maximum likelihood methods and the significance of association can be tested by likelihood ratio tests. In addition, the pedigree-based regression may or may not be corrected or adjusted for one or more factors. The factors for which the analyses may be adjusted include, but are not limited to age, sex, body-mass index, weight, ethnicity, geographic location, fasting state, state of pregnancy or post-pregnancy, menstrual cycle, general health of the subject, alcohol or drug consumption, caffeine or nicotine intake and circadian rhythms, to name a few. If discreet outcome traits are used, a threshold model my be assumed and the analysis can be carried out using the Sequential Oligogenic Linkage Analysis Routines (SOLAR) software program, see Almasy and Warren, Human Genomics, Vol. 2, p 191-195 (2005), which is hereby incorporated by reference.

In unrelated patients, increased risk can be determined from p-values that are derived using logistic regression. Specifically, associations with single nucleotide polymorphisms can be performed by regressing the effect of the marker genotype. Binomial (or binary) logistic regression is a form of regression which is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a logit variable (the natural log of the odds of the dependent occurring or not). In this way, logistic regression estimates the probability of a certain event occurring. These analyses are conducted with the program SAS.

SAS (“statistical analysis software”) is a general purpose package (similar to Stata and SPSS) created by Jim Goodnigt and N.C. State University colleagues. Ready-to-use procedures handle a wide range of statistical analyses, including but not limited to, analysis of variance, regression, categorical data analysis, multivariate analysis, survival analysis, psychometric analysis, cluster analysis, and nonparametric analysis.

The occurrence of pairs of specific alleles at different loci on the same chromosome is not random and the deviation from random is referred to as linkage disequilibrium. Association studies focus on population frequencies and rely on the phenomenon of linkage disequilibrium. If, or example, a specific allele associated with a given gene is directly involved in causing a particular trait, its frequency will be statistically increased in an affected (trait positive) population, when compared to the frequency in a trait negative population or in a random control population. As a consequence of the existence of linkage disequilibrium, the frequency of all other alleles present in the haplotype carrying the trait-related allele should also be increased in trait positive individuals compared to trait negative individuals or random controls. Association between the trait and any other allele in linkage disequilibrium with the trait-causing allele may therefore suggest the presence of a trait-related gene in that particular region of the chromosome. Case-control populations can thus be genotyped for markers to identify associations that narrowly locate a trait causing allele, because an allele in linkage disequilibrium with a given marker associated with a trait of an obesity disorder may be associated with the trait or the obesity disorder itself. Linkage disequilibrium studies allow the relative frequencies in case-control populations of a limited number of genetic polymorphisms to be analyzed as an alternative to screening all possible functional polymorphisms to find trait-causing alleles.

Association studies compare the frequency of marker alleles in unrelated case-control populations, and represent powerful tools for the dissection of complex traits. Population-based association studies do not concern familial inheritance, but instead compare the prevalence of a particular genetic marker, or a set of markers, in case-control populations. These association studies are case-control studies based on a comparison of unrelated trait positive individuals and unrelated control, trait negative individuals. The terms “trait positive population”, “case population” and “affected population” are used interchangeably herein, and the terms “trait negative population”, “control population” and “unaffected population” are used interchangeably herein. Further, the control group may or may not be ethnically matched to the case population. Moreover, the control group may also be matched to the case-population for the main known confusion factor for the trait under study for example, diabetes type II. Ideally, individuals in the two samples are paired in such a way that they are expected to differ only in their trait or disorder status.

One important aspect using association studies is the correct choice for the control populations, and, in turn, affecting this choice is the definition of a given trait or phenotype or disorder. Accordingly, in one embodiment of the present invention, the disorder or trait of a disorder is defined by clinically accepted standards and protocols. In another embodiment, the definition of a disorder or trait of a disorder is the definition that is generally accepted by the scientific community. For example, type 2 diabetes can be defined in the present invention by the standards set forth in the “Report of the Expert Committee on the Diagnosis and Classification of Diabetes Mellitus,” Diabetes Care, Vol. 20:1183-97 (1997), which is hereby incorporated by reference. The association method can be used to analyze a genetic trait if the individuals to be included in the trait positive and trait negative phenotypic groups are carefully selected. In one embodiment, one of four criteria may be used to classify subjects to a control or case population. Examples of the criteria used to classify populations include, but are not limited to, clinical phenotype, age at onset, family history and severity. The selection procedure for continuous traits, e.g., blood pressure, may involve selecting individuals at opposite ends of the phenotype distribution of the trait under study, so as to include in these trait positive and trait negative population individuals with non-overlapping phenotypes. In one embodiment, the case-control populations comprise phenotypically homogeneous populations. In one specific embodiment, trait positive and trait negative populations may comprise phenotypically uniform populations of individuals representing each between about 1 and about 98%. In a more specific embodiment, the populations represent individuals each between about 1 and about 80%, more specifically between about 1 and about 50%, and even more specifically between about 1 and about 30%. In another specific embodiment, the populations represent between about 1 and about 20% of the total population under study, and the population is selected among individuals exhibiting non-overlapping phenotypes. The clearer the difference between the two trait phenotypes, the greater the probability of detecting an association with the marker. The selection of phenotypes that are relatively uniform but drastically different between groups enables efficient comparisons in association studies. Further proper selection of phenotypes allows for the possible detection of marked differences at the genetic level, provided that the sample sizes of the populations under study are significant enough.

The general strategy to perform association studies using the markers described in the present invention was to scan two groups of individuals (case populations and control populations) to determine frequencies of the markers of the present invention in both groups.

If a statistically significant association with a trait or disorder is identified for at least one or more of the analyzed markers, one may be able to assume that either the associated marker is directly responsible for causing the trait or disorder, or that the associated marker may be in linkage disequilibrium with the true trait- or disorder-causing gene or allele. The specific characteristics of the associated marker with respect to the candidate gene function may provide further insight into the relationship between the associated marker and the trait (causal or in linkage disequilibrium). If the evidence indicates that the associated marker within the candidate gene is most probably not the trait-causing gene or allele but is in linkage disequilibrium with the true trait-causing gene or allele, then the trait-causing gene or allele may possibly be found by sequencing the vicinity of the associated marker.

In one embodiment, the association methods are performed in two steps. In a first phase, the frequency of at least one marker is determined in the trait positive and trait negative populations. In a second phase of the analysis, the position of the genetic loci responsible for the given trait is further refined using a higher density of markers from the relevant region. In this manner, the present invention provides methods of identifying disorder-causing genes or alleles of genes in a subject or population of subjects.

The association studies described above, single point studies, can be combined with multi-point association studies, which are referred to as haplotype studies. Haplotype studies may increase the statistical power of single point association studies. As used herein, a haplotype is a set of closely linked markers on one chromosome. The markers of a haplotype may or may not be in linkage disequilibrium.

In a first stage of a haplotype frequency analysis, the frequency of the possible haplotypes based on various combinations of the identified markers of the invention is determined. The haplotype frequency can then be compared for distinct populations of trait positive and control individuals. The number of trait positive individuals and control individuals that are subjected to haplotype frequency analysis can be any number necessary to obtain significant results. In one embodiment, the number of individuals can be between about 30 and about 300 for each population. In another embodiment, the number of individuals used in the haplotype analysis is between about 50 and about 150 for each population. For each evaluated haplotype frequency, a p-value and an odds ratio are calculated, and the results of this first analysis provide haplotype frequencies in case and control populations. If a statistically significant association is found, the relative risk for an individual to possess or develop the trait or disorder can then be approximated.

The markers of the present invention may also be used to identify patterns of markers associated with detectable traits resulting from polygenic interactions. The analysis of genetic interaction between markers at unlinked loci requires individual genotyping using the techniques described herein. The analysis of polygenic interaction based upon a selected set of markers with appropriate level of statistical significance can be considered as a haplotype analysis for the purposes of the present invention. Interaction analysis may comprise stratifying the case-control populations with respect to a given haplotype for the first loci and performing a haplotype analysis with the second loci with each subpopulation.

As mentioned, the invention provides methods for determining an increased risk of acquiring a trait of an obesity disorder or an obesity disorder in a subject. As used herein, the term “subject” is used interchangeably with the term “patient,” and is used to mean an animal, in particular a mammal, and even more particularly a non-human or human primate. Further, the term “acquire” is used to indicate that the trait or obesity disorder becomes visible or detectable in a subject. Thus, if performing the methods of the invention indicates that a patient has an increased risk of acquiring, for example, type 2 diabetes, the intended meaning is that the patient has an increased risk of exhibiting one or more signs or symptoms of type 2 diabetes.

It will of course be understood by practitioners skilled in the treatment or diagnosis of obesity disorders that the present invention may not provide an absolute identification of individuals who could be at risk of developing a particular disorder or a trait of a disorder. Rather, the present invention may indicate a certain degree or likelihood of developing an obesity disorder or a trait of an obesity disorder. The information relating to the likelihood of developing a disorder, however, is extremely valuable in that the information may initiate preventive treatments or to allow an individual carrying a marker to foresee warning signs and/or minor symptoms. In diseases in which attacks may be extremely violent and sometimes fatal if not treated in time, the knowledge of a potential predisposition, even if this predisposition is not absolute, might contribute in a very significant manner to treatment efficacy.

The methods herein relate to determining the increased risk of a subject acquiring or developing an obesity disorder or a trait of an obesity disorder. As used herein, an obesity disorder can be any disorder associated with obesity, and examples include but are not limited to, atherosclerosis, insulin resistance, type 2 diabetes, hypertension, hyperlipidemia, hypertriglyceridemia, cardiovascular disease, microangiopathy in obese individuals with type 2 diabetes, ocular lesions associated with microangiopathy in obese individuals with type 2 diabetes, renal lesions associated with microangiopathy in obese individuals with type 2 diabetes, metabolic syndrome, syndrome X, a fatty liver, fatty liver disease, polycystic ovarian syndrome, hemochromatosis and acanthosis nigricans.

The terms “trait” and “phenotype” are used interchangeably herein and refer to any visible, detectable or otherwise measurable property of an organism such as symptoms of or susceptibility to a disorder, for example. Typically the terms “trait” or “phenotype” are used herein to refer to symptoms of an obesity disorder, or a susceptibility to an obesity disorder. Examples of traits of obesity disorders include but are not limited to high total cholesterol, low high-density lipoprotein (HDL) cholesterol, impaired fasting glucose levels, insulin resistance, hyperproinsulinemia, central obesity, elevated triglyceride levels, postprandial glucose levels, elevated uric acid levels, thyroid dysfunction, increased body-mass index (BMI), hypertension, impaired glucose tolerance, alterations in hormone and peptide levels (e.g., leptin, ghrelin, obstatin, adiponectin, perilipin, omentin), interactions with substances involved in insulin signaling, lipid, amino acid and glucose metabolism, life expectancy, increased systemic inflammatory state (e.g., as reflected in levels of C-reactive protein, interleukin-6, and TNF-alpha), depression, and sleep disordered breathing.

Examples of additional traits of obesity or obesity disorders include, but are not limited to those listed in Table 1 below.

TABLE 1
Traits of Obesity or Obesity-Related Disorders

| TRAIT ID | Trait Name | Trait Type |
|---|---|---|
| SBP | Systolic Blood Pressure | Continuous |
| DBP | Diastolic Blood Pressure | Continuous |
| TCHOL | Total Cholesterol | Continuous |
| HDL | HDL cholesterol | Continuous |
| FASTG | Fasting Glucose | Continuous |
| GLUO | Glucose at 0 minute | Continuous |
| GLU30 | Glucose at 30 minutes | Continuous |
| GLU60 | Glucose at 60 minutes | Continuous |
| GLU90 | Glucose at 90 minutes | Continuous |
| GLU120 | Glucose at 120 minutes | Continuous |
| GLU150 | Glucose at 150 minutes | Continuous |
| GLU180 | Glucose at 180 minutes | Continuous |
| LNFI | Natural log of Fasting Insulin | Continuous |
| LNIO | Natural log of Insulin at 0 minute | Continuous |
| LNI30 | Natural log of Insulin at 30 minutes | Continuous |
| LNI60 | Natural log of Insulin at 60 minutes | Continuous |
| LNI90 | Natural log of Insulin at 90 minutes | Continuous |
| LNI120 | Natural log of Insulin at 120 minutes | Continuous |
| LNI150 | Natural log of Insulin at 150 minutes | Continuous |
| LNI180 | Natural log of Insulin at 180 minutes | Continuous |
| GABS | Glucose Absorption | Continuous |
| GAUC | Glucose Area Under Curve | Continuous |
| GRESP | Glucose responsiveness | Continuous |
| ISECR | Insulin secretion | Continuous |
| IAUC | Insulin area under the curve | Continuous |
| IRESP | Insulin response | Continuous |
| LIRESP | Natural log of insulin response | Continuous |
| LNTG | Natural log of triglycerides | Continuous |
| BMI | Body mass index | Continuous |
| HOMAIR | Homeostasis model assessment- | Continuous |
| | estimated - insulin resistance | |
| WHR | Waist-Hip Ratio | Continuous |
| LEPTIN | Leptin | Continuous |
| LNLEPT | Natural log of Leptin | Continuous |
| LPTBMI | Leptin adjusted for BMI | Continuous |
| 10WHR | Waist-Hip Ratio * 10 | Continuous |
| LNRES | Natural log of Restraint | Continuous |
| LNDIS | Natural log of Disinhibition | Continuous |
| LNHUN | Natural log of Hunger | Continuous |
| TSH | Thyroid stimulating hormone | Continuous |
| antiTPO | anti TPO antibody | Continuous |
| WAIST | Waist circumference | Continuous |
| DIABO1 | diabetes | Discreet |
| IGTO1 | Impaired glucose tolerance | Discreet |
| DIABIGTO1 | Diabetes/IGT | Discreet |
| HBP01 | Hypertension | Discreet |
| DBPHILO | DBP | Discreet |
| METSYND | Metabolic Syndrome | Discreet |

The term “genotype” as used herein refers to the identity of the collection of alleles or markers present in an individual or a sample. In the context of the present invention, a genotype generally refers to the description of the markers present in an individual or a sample. The term “genotyping” a sample or an individual for a marker comprises determining the specific marker or the specific nucleotide sequence that an individual is carrying.

The term “polymorphism” as used herein refers to the occurrence of two or more alternative genomic sequences or alleles between or among different genomes or individuals. “Polymorphic” thus refers to the condition in which two or more variants or markers of a specific genomic sequence can be found in a population. A “polymorphic site” is the locus at which the variation occurs. A single nucleotide polymorphism (SNP) is a single base pair change. As used herein, an SNP can be the replacement of one nucleotide by another nucleotide at a non-polymorphic site or a polymorphic site. Of course, deletion of a single nucleotide or insertion of a single nucleotide, also gives rise to single nucleotide polymorphisms. In one embodiment of the present invention, a “single nucleotide polymorphism” is a single nucleotide substitution. The polymorphic site may be occupied by two different nucleotides between different genomes or between different individuals. In other embodiment, the SNP involves an insertion or a deletion of at least one nucleotide. In specific embodiments, the SNP involves an insertion or deletion of between about 1 and about 8 nucleotides.

The term “biallelic polymorphism” refers to a polymorphism having two alleles or markers at a fairly high frequency in the population. A “biallelic marker” refers to the nucleotide variants present at a biallelic polymorphic site. In one embodiment, the frequency of the less common allele of the biallelic markers of the present invention are validated to be greater than about 1%, greater than about 10%, greater than about 15%, greater than about 20% and greater than about 30%. The alleles or markers need not, however, conform to Hardy-Weinberg equilibrium for the purposes of the present invention.

As used herein the term “TR-related biallelic marker” relates to a set of biallelic markers in linkage disequilibrium with a taste receptor. Unless otherwise specified, the term “TR-related biallelic marker” embraces both validated and non-validated biallelic markers in linkage disequilibrium with a taste receptor.

The methods comprise determining a test genetic sequence of at least one taste receptor gene in a test subject. As used herein, the phrase “test genetic sequence” refers to an unknown polynucleotide sequence of a nucleic acid taken from the subject to be tested. Methods of assessing the genetic sequence of polynucleotides are well known in the art, and the invention is not limited to the type of protocols employed to determine polynucleotide sequences of a given segment of nucleic acid. The nucleic acids to be sequenced can be any nucleic acids including DNA, RNA and even DNA/RNA hybrid sequences. The nucleic acids can be single-stranded or double-stranded prior to sequencing.

The genomic DNA samples from which the genetic sequences are determined may be obtained from related individuals or unrelated individuals corresponding to a heterogeneous population of known ethnic background. The number of individuals from whom DNA samples are obtained can vary substantially, from about 10 or less to about 1000 or more. In one embodiment, the number of individuals from which sample DNA is obtained is between about 50 to about 500 individuals. It is usually desirable to collect DNA samples from at least about 100 individuals to have sufficient polymorphic diversity in a given population to identify as many markers as possible and to generate significant results.

The invention is not limited by the type of test sample or the source of the genomic DNA that is sequenced. Examples of test samples or sources of DNA include, but are not limited to, biological fluids, which can be tested by the methods of the present invention described herein, and include but are not limited to whole blood, serum, plasma, cerebrospinal fluid, urine amniotic fluid, lymph fluids, and various external secretions of the respiratory, intestinal and genitourinary tracts, tears, saliva, milk, white blood cells, myelomas and the like. Samples to be assayed also tissue specimens including tumor and non-tumor tissue and lymph node tissues, bone marrow aspirates and even fixed cell specimens. Techniques to prepare genomic DNA from biological samples are well known to the skilled technician. The person skilled in the art can choose to amplify pooled or unpooled DNA samples.

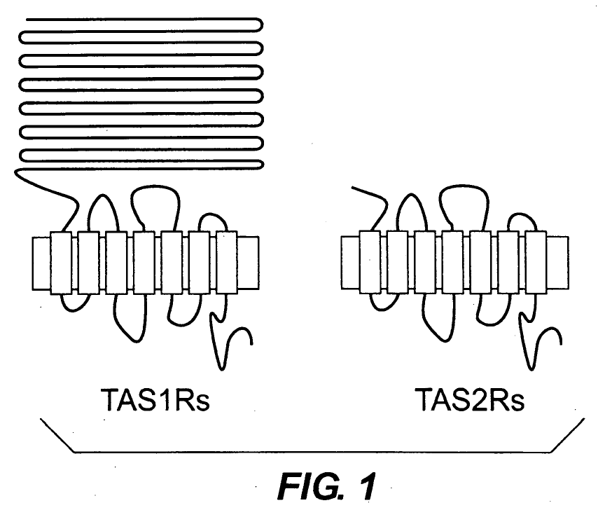

The methods of the present invention comprise sequencing at least one taste receptor gene from a test individual. As used herein, a taste receptor is used as it is in the art. Namely, taste receptors are encoded by genes that generally fall within the TAS1R or TAS2R receptor families and have the general structure as shown in FIG. 1. As mentioned above, TAS1R receptor genes code for taste receptors for the sweet and umami tastes and are generally comprised of large extracellular N-terminal domains and transmembrane domains, and are G-protein-coupled receptor proteins. Currently, there are three known TAS1R receptor genes, but the invention could be applied to later-discovered TAS1R genes and SNPs thereof. The TAS2R receptor genes, on the other hand, code for receptors for the bitter taste and are generally comprised of G-protein coupled receptor proteins. Currently, there are about 30 TAS2R genes, but the invention should not be limited to later-discovered genes and SNPs thereof.

DNA amplification methods may or may not be employed in the methods of the present invention. DNA samples can be pooled or unpooled for the amplification step. While the amplification of target or signal is often desirable, ultrasensitive detection methods which do not require amplification are also encompassed by the present genotyping methods. DNA amplification techniques are well known to those skilled in the art, and include but are not limited to, PCR (polymerase chain reaction) methods or by developments or alternatives thereof. Amplification methods which can be utilized herein include, but are not limited to, Ligase Chain Reaction (LCR), nucleic acid sequence-based amplification (NASBA), self-sustained sequence replication (3SR), Q-beta amplification, strand displacement amplification, and target mediated amplification to name a few. Some of these amplification methods may be particularly suited for the detection of SNPs and allow the simultaneous amplification of a target sequence and the identification of the poly orphic nucleotide.

LCR and Gap LCR are exponential amplification techniques, and both depend on DNA ligase to join adjacent primers annealed to a DNA molecule. In an LCR assay, probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′-3′ relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes which can be ligated to form a complementary, secondary ligated product. Through repeated cycles of hybridization and ligation, amplification of the target sequence can be achieved. Methods of LCR also include multiplex LCR, Cap LCR (GLCR) is simply a version of LCR where the probes are not adjacent but are separated by about 2 to 3 bases. Asymmetric Cap LCR (RT-AGLCR) is a modification of GLCR that allows the amplification of RNA.

For amplification of mRNAs, it is within the scope of the present invention to reverse transcribe mRNA into cDNA followed by polymerase chain reaction (RT-PCR). Alternatively, a single enzyme may be used for both steps as described in U.S. Pat. No. 5,322,770, which is hereby incorporated by reference.

In one embodiment, PCR technology is used in the present invention to amplify the DNA sample. A variety of PCR techniques are familiar to those skilled in the art. For a review of PCR technology, see White, B. A. (1997) and the publication entitled “PCR Methods and Applications” (1991, Cold Spring Harbor Laboratory Press), both of which are hereby incorporated by reference. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, several cycles of denaturation, hybridization, and extension are initiated to produce an amplified fragment containing the nucleic acid sequence between the primer sites.

Amplification can be performed using the primers initially used to discover new markers, or any set of primers allowing the amplification of a DNA fragment comprising a marker of the present invention. Primers can be prepared by any suitable method, such as, but not limited to direct chemical synthesis by a method such as the phosphodiester, the phosphodiester method, the diethylphosphoramidite, and the solid support method.

The amplified or unamplified DNA can be sequenced using any method known and available to the skilled technician. The entire DNA sample need not be sequenced, provided that the polynucleotide sequence comprising the marker of interest is sequenced such that a SNP is identifiable. Accordingly, the phrase “genetic sequence of at least one taste receptor” is not intended to mean that the entire polynucleotide sequence of the taste receptor gene in question be sequenced. Methods for sequencing DNA using either the dideoxy-mediated method (Sanger method) or the Maxam-Gilbert method are widely known to those of ordinary skill in the art. Such methods are disclosed, for example, in Sambrook et al. (1989), which is hereby incorporated by reference. Alternative approaches include hybridization to high-density DNA probe arrays as described in Chee et al. (1996), which is hereby incorporated by reference.

In one embodiment, the DNA is sequenced by automated dideoxy terminator sequencing reactions using a dye-primer cycle sequencing protocol. The products of the sequencing reactions can be run on sequencing gels and the sequences are determined using gel image analysis. In this embodiment, the polymorphism search is based on the presence of superimposed peaks in the electrophoresis pattern resulting from different bases occurring at the same position. Because each dideoxy terminator is labeled with a different fluorescent molecule, the two peaks corresponding to a biallelic site present distinct colors corresponding to two different nucleotides at the same position on the sequence. The presence of two peaks can be a SNP or it may be an artifact due to background noise. To exclude such an artifact, the two strands of the double-stranded nucleic acid can be sequenced and a comparison between the peaks can be carried out. The SNP would then need to be detected on both strands.

The detection limit for the frequency of biallelic polymorphisms detected by sequencing pools of 100 individuals is approximately 0.1 for the minor allele, as verified by sequencing pools of known allelic frequencies. More than 90% of the biallelic polymorphisms that are detected by the pooling method have a frequency for the minor allele higher than 0.25. In one embodiment, the biallelic markers selected by the pooled dideoxy terminator sequencing reactions have a detection limit of about 0.1 for the minor allele and less than about 0.9 for the major allele. In a more specific embodiment, the detection limit is about at least 0.2 for the minor allele and less than about 0.8 for the major allele. In an even more specific embodiment, the limits are at least about 0.3 for the minor allele and less than about 0.7 for the major allele.

In one embodiment, the method of identifying SNPs of at least one taste receptor of the present invention comprises reviewing databases of known SNPs at known loci on a chromosome or within a gene. As such, the SNPs may be identifiable by a reference identification number (a reference ID) in publicly or commercially available SNP databases. In one embodiment, the SNP database that is searched is the dbSNP database available through the United States National Institutes of Health, National Center for Biotechnology information (NCBI) and is available on the World Wide Web at www.ncbi.nlm.nih.gov/projects/SNP/ or at www.ncbi.nlm.nih.gov/entrez/query fcgi?db=snp. In another embodiment, a commercially available database though Celera Genomics in Rockville, Md., USA may also be searched.

Examples of known SNPs within the TAS1R or TAS2R genes include, but are not limited to SNPs identified in TAS1R1, TAS1R2, TAS1R3 (see Kim et al 2006) which is hereby incorporated by reference), SNPs identified in TAS2R1, TAS2R2, TAS2RS, TAS2R4, TAS2R5, TAS2R7, TAS2R8, TAS2R9, TAS2R10, TAS2R13, TAS2R14, TAS2R16, TAS2R38, TAS2R39, TAS2R40, TAS2R41, TAS2R43, TAS2R44, TAS2R45, TAS2R46, TAS2R47, TAS2R48, TAS2R49, TAS2R50, TAS2R55, and TAS2R60 (see Kim et al. 2003, Kim et al. 2005 and Wang et al. 2004, which are hereby incorporated by reference), and SNPs identifiable by a reference identification number in publicly or commercially available SNP databases as discussed above. In one particular embodiment, SNPs within or neat TAS2R genes include, but are not limited to, rs4726600, rs10278721, rs4595035, rs10241042, rs534126, rs5020531, rs3741845, rs2588350 and rs10772420. In another embodiment, the SNPs include mutations that change the coding sequence of the encoded peptide, such as, but not limited to, A49P, V262A, and 1296V of TAS2R38. To be clear, the “A49P” nomenclature indicates that an alanine at amino acid position 49 of the peptide is mutated to a proline residue. In another particular embodiment, SNPs within or near TAS1R genes include, but are not limited to, rs12036097, rs12567264, and rs12408808.

In another embodiment, methods of identifying SNPs involve directly determining the identity of the nucleotide present at a marker site by sequencing assays, allele-specific amplification assays, or hybridization assays. Methods well-known to those skilled in the art that can be used to detect polymorphisms include methods such as, conventional dot blot analyzes, single strand conformational polymorphism analysis (SSCP) described by Orita et al. (1989), denaturing gradient gel electrophoresis (DGGCE), heteroduplex analysis, mismatch cleavage detection, and other conventional techniques as described in Sheffield et al. (1991), White et al. (1992), Grompe et al. (1989 and 1993). Another method for determining the identity of the nucleotide present at a particular site employs a specialized exonuclease-resistant nucleotide derivative as described in U.S. Pat. No. 4,656,127, which is hereby incorporated by reference.

In microsequencing methods, the nucleotide at a particular site in a target DNA is detected by a single nucleotide primer extension reaction. This method involves appropriate microsequencing primers which hybridize just upstream of the particular base of interest in the target nucleic acid. A polymerase is used to specifically extend the 3′ end of the primer with one single ddNTP (chain terminator) complementary to the nucleotide at the polymorphic site. Next the identity of the incorporated nucleotide is determined in any suitable way.

Typically, microsequencing reactions are carried out using fluorescent ddNTPs and the extended microsequencing primers are analyzed by electrophoresis on ABI 377 sequencing machines to determine the identity of the incorporated nucleotide. Alternatively capillary electrophoresis can be used to process a higher number of assays simultaneously.

Different approaches can be used for the labeling and detection of ddNTPs. A homogeneous phase detection method based on fluorescence resonance energy transfer has been described by Chen and Kwok (1997) and Chen et al. (1997). In this method, amplified genomic DNA fragments containing particular sites are incubated with a 5′-fluorescein-labeled primer in the presence of allelic dye-labeled dideoxyribonucleoside triphosphates and a modified Taq polymerase. The dye-labeled primer is extended one base by the dye-terminator specific for the allele present on the template. At the end of the genotyping reaction, the fluorescence intensities of the two dyes in the reaction mixture are analyzed directly without separation or purification. All the steps in this genotyping reaction can be performed in the same tube and the fluorescence changes can be monitored in real time. Alternatively, the extended primer may be analyzed by MALDI-TOF Mass Spectrometry. The base at the polymorphic site is identified by the mass added onto the microsequencing primer (see Haff and Smirnov, 1997).

Microsequencing may be achieved by the established microsequencing method or by developments or derivatives thereof. Alternative methods include several solid-phase microsequencing techniques. The basic microsequencing protocol is the same as described previously, except that the method is conducted as a heterogeneous phase assay, in which the primer or the target molecule is immobilized or captured onto a solid support. To simplify the primer separation and the terminal nucleotide addition analysis, oligonucleotides are attached to solid supports or are modified in such ways that permit affinity separation as well as polymerase extension. The 5′ ends and internal nucleotides of synthetic oligonucleotides can be modified in a number of different ways to permit different affinity separation approaches, e.g., biotinylation. If a single affinity group is used on the oligonucleotides, the oligonucleotides can be separated from the incorporated terminator reagent, which eliminates the need of physical or size separation. More than one oligonucleotide can be separated from the terminator reagent and analyzed simultaneously if more than one affinity group is used, which permits the analysis of several nucleic acid species or more nucleic acid sequence information per extension reaction. The affinity group need not be on the priming oligonucleotide, but the group could alternatively be present on the template. For example, immobilization can be carried out via an interaction between biotinylated DNA and streptavidin-coated microtitration wells or avidin-coated polystyrene particles. In the same manner, oligonucleotides or templates may be attached to a solid support in a high-density format. In such solid phase microsequencing reactions, incorporated ddNTPs can be radiolabeled (Syvanen, 1994) or linked to fluorescein (Livak and Hainer, 1994). The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such as p-nitrophenyl phosphate). Other possible reporter-detection pairs include: ddNTP linked to dinitrophenyl (DNP) and anti-DNP alkaline phosphatase conjugate (Harju et al., 1993) or biotinylated ddNT and horseradish peroxidase-conjugated streptavidin with o-phenylenediamine as a substrate. As yet another alternative solid-phase microsequencing procedure, Nyen et al. (1993) described a method relying on the detection of DNA polymerase activity by an enzymatic luminometric inorganic pyrophosphate detection assay (ELIDA).

Pastinen et al. (1997) describe a method for multiplex detection of single nucleotide polymorphism in which the solid phase microsequencing principle is applied to an oligonucleotide array format. High-density arrays of DNA probes attached to a solid support (DNA chips) are further described below. It will be appreciated that any primer having a 3′ end immediately adjacent to the polymorphic nucleotide may be used. Similarly, it will be appreciated that microsequencing analysis may be performed for any marker or any combination of biallelic markers of the present invention.

In one aspect the present invention provides polynucleotides and methods to determine the allele or an SNP of one or more biallelic markers or SNPs of the present invention in a biological sample, by mismatch detection assays based on the specificity of polymerases and/or ligases. Polymerization reactions places particularly stringent requirements on correct base pairing of the 3′ end of the amplification primer and the joining of two oligonucleotides hybridized to a target DNA sequence is quite sensitive to mismatches close to the ligation site, especially at the 3′ end.

Discrimination between the two alleles of a biallelic marker can also be achieved by allele specific amplification, a selective strategy, whereby one of the alleles is amplified without amplification of the other allele. This selective amplification is accomplished by placing the polymorphic base at the 3′ end of one of the amplification primers. Because the extension forms from the 3′ end of the primer, a mismatch at or near this position has an inhibitory effect on amplification. Therefore, under appropriate amplification conditions, these amplification primers only direct amplification on their complementary allele. Designing the appropriate allele-specific primer and the corresponding assay conditions are well with the ordinary skill in the art.

The “Oligonucleotide Ligation Assay” (OLA) uses two oligonucleotides which are designed to be capable of hybridizing to abutting sequences of a single strand of at target molecule. One of the oligonucleotides is biotinylated, and the other is detectably labeled. If the precise complementary sequence is found in a target molecule, the oligonucleotides will hybridize such that their termini abut and create a ligation substrate that can be captured and detected. OLA is capable of detecting SNPs and may be combined with PCR as described by Nickerson D. A, et al. (1990). This combination OLA and PCR methodology can be used to achieve the exponential amplification of target DNA, which is then detected using OLA.

Other methods which are particularly suited for the detection of single nucleotide polymorphism include LCR (ligase chain reaction), Gap LCR (GLCR) which are described above. As mentioned above LCR uses two pairs of probes to exponentially amplify a specific target. The sequences of each pair of oligonucleotides are selected to permit the pair to hybridize to abutting sequences of the same strand of the target. Such hybridization forms a substrate for a template-dependant ligase. In accordance with the present invention, LCR can be performed with oligonucleotides having the proximal and distal sequences of the same strand of a biallelic marker site. In one embodiment, either oligonucleotide will be designed to include the biallelic marker site. In such an embodiment, the reaction conditions are selected such that the oligonucleotides can be ligated together only if the target molecule either contains or lacks the specific nucleotide that is complementary to the biallelic marker on the oligonucleotide. In an alternative embodiment, the oligonucleotides will not include the biallelic marker, such that when they hybridize to the target molecule, a “gap” is created. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

Ligase/Polymerase-mediated Genetic Bit Analysis is another method for determining the identity of a nucleotide at a preselected site in a nucleic acid molecule (See WO 95/21271, which is hereby incorporated by reference). The genetic bit analysis method involves the incorporation of a nucleoside triphosphate that is complementary to the nucleotide present at the preselected site onto the terminus of a primer molecule, and its subsequent ligation to a second oligonucleotide. The reaction is monitored by detecting a specific label that is attached to the solid phase of the reaction or by detection in solution.

Another method of determining the identity of the nucleotide present at a biallelic marker site involves nucleic acid hybridization. Any hybridization assay may be used including Southern hybridization, Northern hybridization, dot blot hybridization and solid-phase hybridization (see Sambrook et al., 1989). Additional examples of hybridization assays include, but are not limited, to the TaqMan assay and a molecular beacon assay. The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., 1995). Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., 1998).

The GC content of the probes used in the hybridization assays of the invention can range between about 10% and about 75%, between about 35% and about 60%, and between about 40% and about 55%. The length of the probes can range from 10, 15, 20, or 30 to at least 100 nucleotides. In one embodiment, the marker is within about 4 nucleotides of the center of the polynucleotide probe, including at the center of the probe. Shorter probes may lack specificity for a target nucleic acid sequence and generally require cooler temperatures to form sufficiently stable hybrid complexes with the template. Longer probes are expensive to produce and can sometimes self-hybridize to form hairpin structures. Methods for the synthesis of oligonucleotide probes are well known in the art and can be applied to the probes of the present invention.

In one embodiment, the probes of the present invention are labeled or immobilized on a solid support. Detection probes can then be generally nucleic acid sequences or uncharged nucleic acid analogs, examples of which are well known in the art. The probe may have to be rendered “non-extendable” in that additional dNTPs cannot be added to the probe. Analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively: the 3′ hydroxyl group simply can be cleaved, replaced or modified.

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (the chip) at selected positions. Each DNA chip can contain thousands to millions of individual synthetic DNA probes arranged in a grid-like pattern and miniaturized to the size of a dime.

Chip hybridization technology has already been applied in such cases as BRCA1, in S. cerevisiae mutant strains, and in the protease gene of HIV-1 virus. Chips of various formats for use in detecting biallelic polymorphisms can be produced on a customized basis by Affymetrix (GeneChip™), Hyseq (HyChip and HyGnostics), and Protogene Laboratories.

In general, array methods employ arrays of oligonucleotide probes that are complementary to target nucleic acid sequence segments from an individual which target sequences include a polymorphic marker. Arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of monomers, i.e., nucleotides. Tiling strategies are further described in PCT application No. WO 95/111995, which is incorporated by reference. In a particular aspect, arrays are tiled for a number of specific, identified biallelic marker sequences. In particular, the array is tiled to include a number of detection blocks, each detection block being specific for a specific marker or a set of markers. For example, a detection block may be tiled to include a number of probes, which span the sequence segment that includes a specific polymorphism. To ensure that the probes are complementary to each allele, the probes are synthesized in pairs differing at the marker site. In addition to the probes differing at the polymorphic base, monosubstituted probes can also be tiled within the detection block. These monosubstituted probes have bases at and up to a certain number of bases in either direction from the polymorphism, substituted with the remaining nucleotides (selected from A, T, G, C and U). The monosubstituted probes provide internal controls for the tiled array to distinguish between actual hybridization and cross-hybridization. Upon completion of hybridization with the target sequence and washing of the array, the array can be scanned to determine the position on the array to which the target sequence hybridizes. The hybridization data from the scanned array is then analyzed to identify which marker or markers are present in the sample. Hybridization and scanning may be carried out as described in PCT application No. WO 92/10092 and WO 95/11995 and U.S. Pat. No. 5,424,186, the disclosures of which are hereby incorporated by reference.

Another technique, which may be used to analyze polymorphisms, includes multicomponent integrated systems, which miniaturize and compartmentalize processes such as PCR and capillary electrophoresis reactions in a single functional device. One example of such technique is disclosed in U.S. Pat. No. 5,589,136, which is hereby incorporated by reference.

Integrated systems can, in one instance, be used when microfluidic systems are used. Microfluidic systems comprise a pattern of microchannels designed onto a glass, silicon, quartz, or plastic wafer included on a microchip. The movements of the samples are controlled by electric, electroosmotic or hydrostatic forces applied across different areas of the microchip to create functional microscopic valves and pumps with no moving parts. Varying the voltage can control the liquid flow at intersections between the micro-machined channels and can change the liquid flow rate for pumping across different sections of the microchip.

For genotyping markers, the microfluidic system may integrate nucleic acid amplification, microsequencing, capillary electrophoresis and a detection method such as laser-induced fluorescence detection.

A technique to identify polymorphisms includes PCR amplification of individual genes or gene fragments and diagnostic digestion with restriction endonucleases. For example, an endonuclease is selected to differentially digest two alleles as the SNP disrupts an endonuclease recognition site or creates a novel endonuclease recognition site. One example of such technique is disclosed in Nelson et al. 2005, which is herein incorporated by reference.

Once the test genetic sequence of at least one taste receptor gene is determined, i.e., the sequence of interest has been genotyped, the test sequence can be reviewed to determine if at least one marker is present that would be indicative of an increased risk of developing a disorder or trait of a disorder. This determination of increased risk may or may not involve reviewing the test genetic sequence for the presence of at least one risk allele. As used herein, a “risk allele” is used to mean a chromosomal major or minor allele associated with a given taste receptor gene that correlates with a trait of an obesity-related disorder or an obesity-related disorder. Alternatively, the determination may or may not involve comparison of the test genetic sequence to a sequence that has a known or accepted correlation with a particular phenotype.

As used herein, “in association with” when used in relation of a SNP to a taste receptor gene, is used to mean in moderate or high linkage disequilibrium (LD) with at least a portion of the coding region of a taste receptor gene. Moderate LD is defined as an r² value of at least 0.4 but no greater than 0.7. High LD is defined as r² value of at least 0.7 and as great as 1.0.

The term “gene” is used similarly to as it is in the art. Namely, a gene is a region of DNA that is responsible for the production and regulation of a polypeptide chain. Genes include both coding and non-coding portions, including introns, exons, promoters, initiators, enhancers, terminators and other regulatory elements. As used herein, “gene” is intended to mean at least a portion of a gene. Thus, for example, “gene” may be considered a promoter for the purposes of the present invention. In a particular embodiment, the non-coding portion of the gene is a promoter. In a particular embodiment, the coding portion of the gene is at the 5′ end of the coding portion of the gene. In another particular embodiment, the coding portion of the gene is at the 3′ end of the coding portion of the gene.

The present invention also provides for methods of determining a statistical association or correlation between a particular genotype and a phenotype. The methods comprise genotyping a trait positive population and a control population and determining if a statistical association or correlation exists between a particular genotype and a phenotype. Several methods of correlating a genotype to a phenotype exist in the art, and the invention is not limited to a particular type of association method. For example, the methods of associating or correlating genotypes to a phenotype include, but are not limited to, parametric and non-parametric analysis.

In one embodiment, a linkage analysis is used to determine a correlation between a given genotype and a trait or disorder. A linkage analysis is based upon establishing a correlation between the transmission of genetic markers and that of a specific trait throughout generations within a family. Thus, the aim of linkage analysis is to detect marker loci that show cosegregation with a trait of interest in pedigrees.

When data are available from successive generations, there is the opportunity to study the degree of linkage between pairs of loci. Estimates of the recombination fraction allow the loci to be ordered and placed onto a genetic map. With markers, a genetic map can be established and then the strength of linkage between the markers and the traits can be calculated and used to indicate the relative positions of markers and genes affecting those traits (Weir, 1996). The classical method for linkage analysis is the logarithm of odds (lod) score method (see Morton, 1955; Ott, 1991). Calculation of lod scores generally requires specification of the mode of inheritance for the disease (parametric method). The length of the candidate region identified using linkage analysis can be between 2 and 20 Mb. Once a candidate region is identified as described above, analysis of recombinant individuals using additional markers allows further delineation of the candidate region. Linkage analysis studies can be used for up to 5,000 microsatellite markers, or perhaps more.

Linkage analysis has been successfully applied to map simple genetic traits that show clear Mendelian inheritance patterns and which have a high penetrance (i.e., the ratio between the number of trait positive carriers of an allele and the total number of a carriers in the population).

Non-parametric methods for linkage analysis do not necessarily require specification of the mode of inheritance for the disease, and they can often be more useful for the analysis of complex traits. In non-parametric methods, there is an attempt to demonstrate that the inheritance pattern of a chromosomal region is not consistent with random Mendelian segregation by showing that affected relatives inherit identical copies of the region more often than would expected if based strictly on chance. Affected relatives should show excess “allele sharing” even in the presence of incomplete penetrance and polygenic inheritance. In non-parametric linkage analysis, the degree of agreement at a marker locus in two individuals can be measured either by the number of alleles identical by state (IBS) or by the number of alleles identical by descent (IBD). Affected sib pair analysis is a well-known special case and is the simplest form of these non-parametric methods.

The markers of the present invention may be used in both parametric and non-parametric linkage analysis. In one embodiment, the markers may be used in non-parametric methods to allow the mapping of genes involved in complex traits. The markers of the present invention may be used in both IBD- and IBS-methods to map genes affecting a complex trait. In such studies several adjacent marker loci may be pooled to take advantage of the high density of markers and to achieve the efficiency attained by multi-allelic markers (Zhao et al., 1998).

Several different approaches can be employed to perform association studies: genome-wide association studies, candidate region association studies and candidate gene association studies. In a preferred embodiment, the markers of the present invention are used to perform candidate gene association studies. The candidate gene analysis clearly provides a short-cut approach to the identification of genes and gene polymorphisms related to a particular trait when some information concerning the biology of the trait is available. Further, the markers of the present invention may be incorporated in any map of genetic markers of the human genome to perform genome-wide association studies. The markers of the present invention may further be incorporated into any map of a specific candidate region of the genome (a specific chromosome or a specific chromosomal segment for example).

Association studies may be conducted within the general population and can also be performed on related individuals in affected families. Association studies are extremely valuable as they permit the analysis of sporadic or multifactor traits. Moreover, association studies represent a powerful method for fine-scale mapping enabling much finer mapping of trait causing alleles than linkage studies. Association studies using the markers of the present invention can therefore be used to refine the location of a trait causing allele in a candidate region identified by linkage analysis methods. Once a chromosome segment of interest has been identified, the presence of a candidate gene or SNP in the region of interest can provide a shortcut to the identification of the trait causing allele or marker. The markers of the present invention, therefore, can be used to demonstrate that a candidate marker is correlated with a trait, and such uses are specifically contemplated in the present invention and claims.

Linkage disequilibrium is the non-random association of markers at two or more loci and represents a powerful tool for mapping genes involved in disease traits (see Ajioka R. S. et al., 1997). Because SNPs can be densely spaced in the human genome and can be genotyped in more numerous numbers than other types of genetic markers (such as RFLP or VNTR markers), SNPs are particularly useful in genetic analysis based on linkage disequilibrium.

When a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation, i.e., the disease mutation is present only in association with a specific set of marker alleles. Through subsequent generations, recombinations may occur between the disease mutation and these marker polymorphisms, and the disequilibrium may gradually dissipate. The pace of this dissipation is a function of the recombination frequency, so the markers closest to the disease gene will manifest higher levels of disequilibrium than those that are further away. When not broken up by recombination, “ancestral” haplotypes and linkage disequilibrium between markers at different loci can be tracked not only through pedigrees but also through populations. Linkage disequilibrium is usually seen as an association between one specific allele at one locus and another specific allele at a second locus.

Haplotype distribution can be synthetically described as:

$$π = \begin{matrix} & B & b & \\ A & x & p - x & p \\ a & q - x & 1 - p - q + x & 1 - p \\ & q & 1 - q & 1\end{matrix} . \qquad ( 1 )$$

Fixing the marginals p and q, the distribution π is completely identified by the probability x of the haplotype (A, B). The discrepancy of a generic π from the distribution under linkage disequilibrium, can be quantified by D=(x−pg).

Measures of LD are defined as the standardized values of D. Two common such measures are

$${R}^{2} = \frac{{( x - pq )}^{2}}{pq ( 1 - p ) ( 1 - q )} and {D}^{′} = \frac{( x - pq )}{D max}$$

where Dmax is min(p(1−q), q(1−p) when the numerator is positive, and min(pq,(1−p)(1−q)) otherwise.

The measure R² ranges between 0 and 1, and it is equal to 1 only when two entries of the table in (1) are equal to 0. The measure D′ ranges, by definition, between −1 and 1, and its absolute value is equal to 1 whenever one entry of the table in (1) is equal to 0.

D′ is a measure of linkage disequilibrium between two genetic markers. A value of D′=1 (complete LD) indicates that two SNPs have not been separated by recombination, while values of D′<1 (incomplete LD) indicate that the ancestral LD was disrupted during the history of the population (only D′ values near one are a reliable measure of LD extent; lower D′ values are usually difficult to interpret as the magnitude of D′ strongly depends on sample size.

R² is a measure of linkage disequilibrium between two genetic markers. For SNPs that have not been separated by recombination or have the same allele frequencies (perfect LD), R²=1. In such case, the SNPs are said to be redundant. Lower R² values indicate less degree of LD.

Typically, R² is preferred when the focus is on the predictability of one polymorphism given the other (and hence it is often used in power studies for association designs). D′, instead, is the measure of choice to assess recombination patterns.

The markers of the present invention may further be used in TDT (transmission disequilibrium test). TDT tests for both linkage and association and is not affected by population stratification. TDT requires data for affected individuals and their parents or data from unaffected sibs instead of from parents (see Spielmann S. et al., 1993; Schaid D. J. et al., 1996, Spielmann S. and Ewens W. J., 1998). Such combined tests generally reduce the false-positive errors produced by separate analyses.

The invention also provides methods of diagnosing an individual with an obesity disorder. As used herein the term “diagnose” means to confirm the results of other tests or to simply confirm suspicions that the subject may have a particular disorder. In other words, the diagnostic tests of the present invention are used in conjunction with other tests, regardless of timing of the other tests. A “test,” on the other hand, is used to indicate a screening method where the subject or the healthcare provider has no indication that the subject may, in fact, have a particular disorder. Thus, a test may be a screening method where a patient exhibits some general symptom. For example, a patient may exhibit a symptom or symptoms that do not clearly indicate a specific disorder. The testing methods described herein could then be used to determine if the subject needs additional diagnostic procedures to properly diagnose the disorder that may be causing the general symptom(s). The methods of testing herein may be used for a definitive diagnosis, or the tests may be used to assess a subject's likelihood or probability of developing a disorder or trait of a disorder.

The methods of testing and diagnosing comprise obtaining the genetic sequence of a particular taste receptor gene and comparing this test sequence to a sequence known to be associated with a particular obesity disorder. The methods of genotyping and comparing are described herein. Furthermore, methods of associating a particular genotype with a particular disorder or trait of a disorder are also described fully herein.

The present invention also provides methods of altering the levels of incretin hormones, e.g., glucagon-like peptide 1 (GLP-1), secreted from enteroendocrine cells. Incretins are well-known peptides that, generally speaking, increase insulin release from beta cells. Examples of incretins include, but are not limited to, GLP-1 and glucose-dependent insulinotropic peptide (GIP). Activation of TAS1R or TAS2R receptors by their cognate ligands can alter enteroendocrine cell function, e.g. by promoting the secretion of incretin hormones such as GLP-1 (Jang et al. 2007; Sternini et al., 2008). The methods comprise administering to the cells a compound that preferably affects the activity of a TAS2R9 taste receptor present on the surface of enteroendocrine cells. Affecting the activity of the TAS2R9 taste receptor will, in turn, alter the secretion of incretin hormones, e.g., GLP-1, from the enteroendocrine cells. In one embodiment, it is desirable to diminish the levels of incretin hormone, e.g., GLP-1, secreted from the enteroendocrine cells, by administering a compound that reduces the activity of the TAS2R9 receptor. In one embodiment, it may be desirable to increase the levels of incretin hormone, e.g., GLP-1, secreted from the enteroendocrine cells, by administering a compound that increases the activity of the TAS2R9 receptor. The methods of altering incretin hormone, e.g., GLP-1, secretion can be performed on any type of TAS2R9 receptor, such as a dominant, recessive, wild-type or mutant receptor.

If the compound is to be administered to a subject, the compounds can be administered as part of a pharmaceutical composition in admixture or mixture with pharmaceutically acceptable carriers.

The dosage of administered agent will vary depending upon such factors as the patient's age, weight, height, sex, general medical condition, previous medical history, etc. In general, it is desirable to provide the recipient with a dosage of compounds that affect TAS1R or TAS2R activity which is in the range of from about 1 pg/kg to 10 mg/kg (body weight of patient), although a lower or higher dosage may be administered. When providing compounds that affect TAS1R or TAS2R activity to a patient, it is preferable to administer such compounds in a dosage which also ranges from about 1 pg/kg to 10 mg/kg (body weight of patient) although a lower or higher dosage may also be administered. In one embodiment, two or more compounds are co-administered to affect the activity of TAS1R or TAS2R receptors. As used herein, compounds are said to be co-administered with when the compounds are administered in such proximity of time that the co-administered compounds can be detected at the same time in the patient's serum.

The compounds that affect TAS1R or TAS2R activity may be administered to patients intravenously, intramuscularly, subcutaneously, enterally, or parenterally. When administering by injection, the administration may be by continuous infusion, or by single or multiple boluses.

The administration compounds that affect TAS1R or TAS2R activity may be for either a “prophylactic” or “therapeutic” purpose. When provided prophylactically, the compounds that affect TAS1R or TAS2R activity are provided in advance of symptom of an obesity-related disorder. The prophylactic administration of the compound(s) serves to prevent or attenuate any subsequent symptom of an obesity related disorder from occurring of progressing. When provided therapeutically, compounds that affect TAS1R or TAS2R activity are provided at (or shortly after) the onset of at least one symptom of an obesity-related disorder.

A composition is said to be “pharmacologically acceptable” if its administration can be tolerated by a recipient patient. Such an agent is said to be administered in a “therapeutically effective amount” if the amount administered is physiologically significant. An agent is physiologically significant if its presence results in a detectable change in the physiology of a recipient patient.

The compounds that affect TAS1R or TAS2R activity can be formulated according to known methods to prepare pharmaceutically useful compositions, whereby these materials, or their functional derivatives, are combined in admixture with a pharmaceutically acceptable carrier vehicle. Suitable vehicles and their formulation are described, for example, in Remington's Science and Practice of Pharmacy (21st ed., Hendrickson, R., et al., Eds., Lippincott Williams & Wilkins, Baltimore, Md. (2006)), which is incorporated by reference. To form a pharmaceutically acceptable composition suitable for effective administration, such compositions will contain an effective amount of compounds that affect TAS1R or TAS2R activity, or their functional derivatives, together with a suitable amount of carrier vehicle.

Additional pharmaceutical methods may be employed to control the duration of action. Control release preparations may be achieved through the use of polymers to complex or absorb compounds that affect TAS1R or TAS2R activity, or their functional derivatives. The controlled delivery may be exercised by selecting appropriate macromolecules (for example polyesters, polyamino acids, polyvinyl, pyrrolidone, ethylenevinylacetate, methylcellulose, carboxymethylcellulose, or protamine, sulfate) and the concentration of macromolecules as well as the methods of incorporation in order to control release. Another possible method to control the duration of action by controlled release preparations is to incorporate compounds that affect TAS1R or TAS2R activity, or their functional derivatives, into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacylate) microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (2006).

Various embodiments of the present invention are demonstrated in the examples below. The examples are meant to be illustrative and are not intended to limit the scope of the invention in any way.

### EXAMPLES

### Example 1

### SNP Mining and Selection

The Human Genome Database (dbSNP) was used to mine SNPs. SNPs that have been experimentally validated by others (e.g., HapMap) and/or SNPs that predict a functional variant were given priority for further evaluation. Further, the International HapMap Project database was used to identify haplotype tagging SNPs within a 20 kilo-base flanking region of the taste receptor genes. In addition, SNPs were chosen which were polymorphic in the CEU cohort (Utah residents with ancestry from northern and western Europe), have an r² cutoff of 0.8 and a mean frequency of 0.15.

SNP Genotyping

SNPs were genotyped in over 1300 samples from the Old Order Amish of Lancaster County (OOA) cohort. Participants in the AFDS, the Old Order Amish of Lancaster, Pa., have a common lifestyle and socioeconomic status, and possess detailed genealogical records dating to the period of their early migration from Europe in the 1700's (Hseuh et al.) Candidate haplotype-tagging SNPs (r²≧0.8) were identified from HapMap (Nature 437:1299-1320 (2005)) and additional SNPs in coding and regulatory regions from the Entrez SNP database (Sherry et al.) and from the literature. In total, 72 TAS1R- and TAS2R-associated SNPs were genotyped from the Amish Family Diabetes Study (AFDS). Forty-seven of these SNPs were polymorphic in the AFDS and passed quality control filters and were subsequently analyzed. Genotyping was carried out on the Applied Biosystem's Taqman platform according to manufacturer's protocols. Briefly, this is a fluorescence based method that involves use of a 5′ nucleoside probe and unique primer. Table 2 below summarizes the results of the initial genotyping analysis for TAS2R haplotype-tagging SNPs on human chromosome 12.

TABLE 2
Genotyping statistics for chromosome 12 TAS2R SNPs tested in the AFDS

| | | | | | | | | | IAUC |
|---|---|---|---|---|---|---|---|---|---|
| Ch, | | | Call | | Major/ | | | GAUC | Association |
| Position | | | Rate | HWE | Minor | | | Association P | on P |
| (kb) | SNP ID | Linked Gene | (%) | P Value | Allele | MAF | SNP Type | Value | Value |
| 12, | rs2586350^{A} | TAS2R7 | 97.3 | 0.679 | C/T | 0.07 | noncoding | 0.0433 | 0.007 |
| 10844 | | | | | | | | | |
| 12, | rs619381^{A} | TAS2R7 | 94.3 | 0.419 | C/T | 0.07 | M304I | 0.049 | 0.81 |
| 10846 | | | | | | | | | |
| 12, | rs3741845^{A} | TAS2R9 | 97.4 | 0.013 | C/T | 0.12 | A187V | 0.0363 | 0.00582 |
| 10853 | | | | | | | | | |
| 12, | rs10845219^{B} | TAS2R10 | 70.6 | 0.254 | C/T | 0.13 | noncoding | N/A | N/A |
| 10869 | | | | | | | | | |
| 12. | rs1063193 | PRR4 | 89.6 | 0.409 | T/C | 0.45 | Q96R | 0.94 | 0.56 |
| 10891 | | | | | | | | | |
| 12, | rs4281556^{A} | PRH1 | 91.1 | 0.380 | A/G | 0.11 | noncoding | 0.037 | 0.34 |
| 10923 | | | | | | | | | |
| 12, | rs1015443^{C} | TAS2R13 | 97.5 | 0.003 | C/T | 0.21 | S259N | N/A | N/A |
| 10952 | | | | | | | | | |
| 12, | rs7138535^{A} | TAS2R14 | 95.4 | 0.1 | T/A | 0.08 | G38G | 0.80 | 0.64 |
| 10983 | | | | | | | | | |
| 12, | rs10772397^{B} | TAS2R50 | 74.6 | 0.057 | T/C | 0.22 | P259P | N/A | N/A |
| 11030 | | | | | | | | | |
| 12, | rs1376251 | TAS2R50 | 97.4 | 0.941 | C/T | 0.25 | C203Y | 0.48 | 0.82 |
| 11030 | | | | | | | | | |
| 12, | rs6488334 | TAS2R50 | 96.5 | 0.197 | C/T | 0.12 | noncoding | 0.64 | 0.58 |
| 11032 | | | | | | | | | |
| 12, | rs10845278^{B} | TAS2R49 | 71.8 | 0.149 | T/C | 0.50 | noncoding | N/A | N/A |
| 11039 | | | | | | | | | |
| 12, | rs7135018^{A} | TAS2R49 | 89.5 | 0.220 | T/C | 0.11 | K79E | 0.70 | 0.69 |
| 11042 | | | | | | | | | |
| 12, | rs7301234 | TAS2R49 | 91.3 | 0.601 | G/A | 0.28 | noncoding | 0.23 | 0.69 |
| 11042 | | | | | | | | | |
| 12, | rs10772408 | TAS2R49 | 94.3 | 0.576 | T/C | 0.40 | noncoding | 0.56 | 0.21 |
| 11043 | | | | | | | | | |
| 12, | rs10772420 | TAS2R48 | 95.6 | 0.122 | A/G | 0.34 | C299R | 0.010 | 0.033 |
| 11066 | | | | | | | | | |
| 12, | rs1868769^{C} | TAS2R48 | 93.4 | 2.04E−18 | A/G | 0.17 | L140L | N/A | N/A |
| 11066 | | | | | | | | | |
| 12, | rs4763235 | TAS2R48 | 96.3 | 0.96 | C/G | 0.25 | noncoding | 0.35 | 0.26 |
| 11067 | | | | | | | | | |
| 12, | rs11612527^{B} | TAS2R44 | 65.2 | 0.656 | T/A | 0.11 | noncoding | N/A | N/A |
| 11073 | | | | | | | | | |
| 12, | rs10845293^{C} | TAS2R44 | 95.3 | 2.50E−88 | A/G | 0.32 | V227A | N/A | N/A |
| 11075 | | | | | | | | | |
| 12, | rs2708381^{A} | TAS2R46 | 92.6 | 0.243 | G/A | 0.11 | W250# | 0.90 | 0.74 |
| 11105 | | | | | | | | | |
| 12, | rs2708380 | TAS2R46 | 97.1 | 0.107 | T/A | 0.39 | L228M | 0.02 | 0.08 |
| 11105 | | | | | | | | | |
| 12, | rs3759245^{C} | TAS2R45 | 93.4 | 0.001 | T/C | 0.12 | C238R | N/A | N/A |
| n.d. | | | | | | | | | |
| 12, | rs28581524 | TAS2R45 | 91.3 | 0.160 | C/G | 0.24 | H210Q | 0.43 | 0.37 |
| n.d. | | | | | | | | | |
| 12, | rs35720106^{C} | TAS2R43 | 96.5 | 1.53E−44 | C/G | 0.24 | T221T | N/A | N/A |
| 11135 | | | | | | | | | |
| 12, | rs2599404 | TAS2R47 | 97.1 | 0.629 | C/A | 0.36 | L252F | 0.01 | 0.08 |
| 11177 | | | | | | | | | |
| 12, | rs1451772^{C} | TAS2R55/42 | 95.7 | 5.27E−06 | T/C | 0.15 | Y265C | N/A | N/A |
| 11230 | | | | | | | | | |
| 12, | rs5020531 | TAS2R55/42 | 96.2 | 0.025 | C/T | 0.25 | S196F | 0.07 | 0.07 |
| 11230 | | | | | | | | | |

Statistical Analysis

SNPs found to be monomorphic in the AFDS (n=9) were not analyzed further. In the OOA samples, Mendelian discrepancies were screened; inconsistencies that were detected in <0.5% of genotypes were removed from analysis. In addition, Hardy Weinberg Equilibrium was tested (χ² analysis) in all samples to determine if the distribution of genotypes was expected compared to observed allele frequencies. Markers showing extreme deviation from Hardy-Weinberg Equilibrium in controls (p<0.001) were eliminated. SNPs with call rates of >90% were also eliminated from further analysis. Single SNP and association analysis was then performed using pedigree-based analysis. These pedigree-based analyses were carried out by regressing the effect of genotype on trait (adjusting for age and sex, and in some cases for BMI). To account for the relatedness among family members, the measured genotype approach was employed for which an estimated likelihood of specific genetic models was established for the pedigree structure. Parameter estimates were obtained by maximum likelihood methods and the significance of association was tested by likelihood ratio tests. Residual familial correlations among related individuals was accounted for by modeling the pedigree structure as a random effect. For discrete outcome traits, a threshold model was assumed and the analyses were carried out using the SOLAR software program (Almasy 2005). Results of these single SNP association analyses were automatically generated and written into a database.

When fewer than ten individuals were homozygous for the minor allele of any particular SNP, these individuals were combined with heterozygous individuals for analysis. Pairwise linkage disequilibrium (LD) between the SNPs and haplotype block analysis was computed using Haploview 4.0 (Barrett et al.). Haplotype blocks were defined by 95% confidence bounds on D′ (Gabriel et al.).

In addition, the haplotype structure of each positional candidate gene may be determined. Briefly, the haplotype structure was determined using the program Haploview (available on the world wide web at www.broad.mit.edu/mpg/haploview/), which uses both the pedigree structure and linkage disequilibrium information to assign haplotypes. Association analyses may be conducted using the haplotype as a single “super allele” using the program HelixTree (available from Golden Helix, Inc., 716 S. 20^{th} Avenue, Suite 102, Bozeman, Mont. 59718, USA, www.goldenhelix.com) and where haplotypes are correlated with obesity (BMI, percent body fat, waist circumference), related traits (leptin, plasma lipids, glucose and insulin levels, and blood pressure) and eating behavior using variance components and/or haplotype-trend regression techniques.

Results of the analysis are shown in Table 3 which shows a table identifying the SNPs in association with TAS2R39, TAS2R40, TAS2R41, and TAS2R60 genes located on chromosome 7, as well as the SNPs in association with TAS2R42 and TAS2R9 genes located on chromosome 12 with the major/minor allele identified, the trait/disease correlated with the SNP, the odds ratio (calculated by chi-square), and p-value.

TABLE 3

| | Major/Minor | | | | | |
|---|---|---|---|---|---|---|
| SNP | Allele | Type | Chr | Gene | Trait/Disease | P value |
| rs11763979 | G/T | 1360 bp | 7 | TAS2R3 | DIAB* | 0.03 |
| | | upstream | | | | |
| rs4595035 | C/T | Arg310Arg | 7 | TAS2R60 | MetSyn | <0.005 |
| rs534126 | C/T | 1091 bp | 7 | TAS2R40 | IGT/DMIGT | <0.04 |
| | | downstream | | | | |
| rs2588350 | C/T | 1074 bp | 12 | TAS2R7 | DIAB* | <0.0007 |
| | | upstream | | | | |
| rs619381 | C/T | Met304Ile | 12 | TAS2R7 | DIAB* | <0.009 |
| rs3741845 | C/T | Ala187Val | 12 | TAS2R9 | DIAB* | <0.005 |
| rs6488334 | T/A | Intronic/ | 12 | PRR4/ | DIAB* | <0.05 |
| | | Intronic/ | | PRH1/ | | |
| | | 933 bp | | TAS2R50 | | |
| | | downstream | | | | |

500 replicates were simulated to determine that the sample size had an alpha level of 0.01, and that, based on the statistical analyses performed herein, there is a greater than about 60% power to detect an effect of a SNP that contributes to about 1% of the total phenotypic variance of a quantitative trait. The power increases to over 92% for a variance component size of 2% and over 99% for a variance component size of 3%.

### Example 2

### Determination of Linkage Disequilibrium of Selected SNPs

The extent of linkage disequilibrium (LD) was determined using the software package Haploview. Haploview generates marker quality statistics, LD information, haplotype blocks, population haplotype frequencies and single marker association statistics. Pedigree data can be loaded as either partially or fully phased chromosomes or as unphased diplotypes in the standard Linkage format. The latter format also allows the user to specify family structure information as well as disease affection or case/control status. Marker information, including name and location is loaded separately. Upon loading a dataset, the software presents to the user a series of marker genotyping quality metrics. These include a check for conformance with Hardy-Weinberg equilibrium, a tally of Mendelian inheritance errors and the percentage of individuals successfully genotyped for that marker. The program filters out markers that fall below a preset threshold for these tests. The user can adjust these thresholds as well as handpick markers to add or remove from the subsequent steps. Haploview calculates several pairwise measures of LD, including r² and D′, which it uses to create a graphical representation. Alternatively, the user may manually select groups of markers for subsequent haplotype analysis.

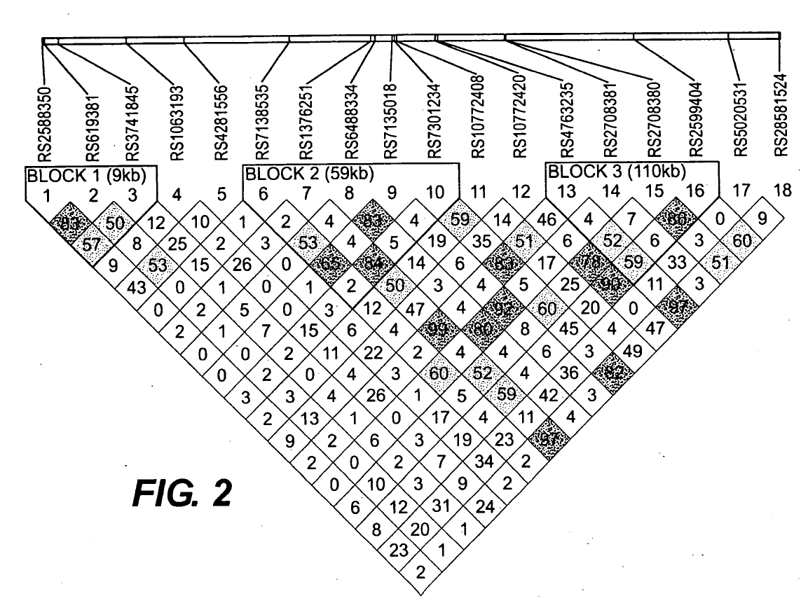

The entire cluster extends for 380 kb and contains three LD blocks of 9 kb, 59 kb and 110 kb (FIG. 2). There was limited LD between TAS2R genes in the AFDS, consistent with what has been reported for other human populations (Kim et al. 2005). SNPs rs2588350 and rs3741845, which share similar associations with glucose and insulin traits and are in close physical proximity, show moderate LD (pairwise r²=0.57) within Block 1. There is minimal LD between the Block 1 SNPs and rs10772420 (pairwise r²=0.09-0.13), suggesting that the association of rs10772420 with glucose and insulin traits could be independent from rs2588350 and rs3741845.

### Example 3

### Association of Taste Receptor Variance with Glucose Homeostasis

To determine whether any taste receptor variants are associated with glucose homeostasis, associations of glucose and insulin areas under the curve (AUC) with SNP genotypes was assessed. A standard 3-hour oral glucose tolerance test (OGTT) (Hseuh et al.) was administered to the 693 non-diabetic AIDS subjects exhibiting the 47 SNPs identified in Example 1. Six TAS2R haplotype-tagging SNPs on chromosome 12 showed significant associations with glucose AUC (Table 2). Three of these SNPs were also significantly associated with insulin AUC as shown in Tables 4 and 5: rs2588350, a noncoding SNP ˜1 kb upstream of the TAS2R gene cluster; rs3741845, a nonsynonymous coding SNP in TAS2R9 (T560C6 encoding Ala187Val); and rs10772420, a nonsynonymous coding SNP in TAS2R48 (A895G, encoding Cys299Arg). No TAS2R-tagging SNPs on chromosomes 5 or 7 were associated with both glucose and insulin AUC, although a single noncoding SNP on chromosome 7, rs534126 showed an association with glucose AUC alone. Surprisingly, no significant association was observed for either glucose AUC or insulin AUC with TAS1R haplotype-tagging SNPs, and all TAS1R3 SNPs were monomorphic in the Amish. Together, these results suggested that one or more TAS2Rs on chromosome 12 impacts glucose homeostasis in non-diabetic individuals.

TABLE 4
Insulin AUC and glucose AUC of three SNPs in the AFDS

| SNP | Trait | | Genotype | | P value |
|---|---|---|---|---|---|
| | | CC | CT/TT | N/A | |
| rs2588350 | Glucose AUC | 19.9 ± 0.2 | 21.3 ± 0.4 | | 0.043 ^{A} |
| (noncoding) | | (n = 600) | (n = 90) | | |
| | Insulin AUC | 739.8 ± 18.0 | 889.8 ± 64.9 | | 0.007 ^{A} |
| | | (n = 593) | (n = 91) | | |
| | | CC | CT/TT | N/A | |
| rs3741845 | Glucose AUC | 19.8 ± 0.2 | 21.0 ± 0.3 | | 0.036 ^{A} |
| (TAS2R9) | | (n = 538) | (n = 155) | | |
| | Insulin AUC | 739.2 ± 19.4 | 858.2 ± 44.2 | | 0.006 ^{A} |
| | | (n = 532) | (n = 155) | | |
| | | AA | AG | GG | |
| rs10772420 | Glucose AUC | 19.6 ± 0.2 | 20.2 ± 0.2 | 21.2 ± 0.4 | 0.01 ^{B} |
| (TAS2R48) | | (n = 324) | (n = 264) | (n = 90) | |
| | Insulin AUC | 718.4 ± 23.2 | 796.5 ± 33.8 | 843.4 ± 51.3 | 0.03 ^{B} |
| | | (n = 323) | (n = 260) | (n = 89) | |

TABLE 5
Age and BMI values, according to genotype,
for AFDS subjects in Table 4

| | | |
|---|---|---|
| SNP/Genotype | Age (yrs) | BMI (kg/m²) |

rs2588350

| | | |
|---|---|---|
| CC (n = 600) | 43.7 ± 0.6 | 26.8 ± 0.2 |
| CT/TT (n = 91) | 45.6 ± 1.4 | 27.4 ± 0.5 |

rs3741845

| | | |
|---|---|---|
| CC (n = 538) | 43.4 ± 0.6 | 26.8 ± 0.2 |
| CT/TT (n = 155) | 46.3 ± 1.1 | 27.1 ± 0.4 |

rs10772420

| | | |
|---|---|---|
| AA (n = 324) | 43.4 ± 0.9 | 26.6 ± 0.3 |
| AG (n = 264) | 44.5 ± 0.9 | 27.1 ± 0.3 |
| GG (n = 90) | 45.3 ± 1.4 | 27.4 ± 0.5 |

To better understand the relationship between these chromosome 12 SNPs and glucose homeostasis, an association analysis of rs2588350, rs3741845 and rs10772420 was extended to other glucose and insulin metrics obtained during the OGTT. The minor allele of each SNP was significantly associated with several measures of glucose and insulin homeostasis (Tables 6, 7 and 8). Subjects with the minor allele for each SNP exhibited higher glucose levels during the first hour of the OGTT; these significantly higher levels persisted into the second hour of the test for those with the minor allele of either rs2588350 or rs10772420. Only subjects with the rs3741845 T (minor) allele showed higher insulin levels in both the first and second hours of the OGTT, although all three SNPs were associated with higher insulin levels for at least one timepoint in the 3 hr test. Estimates of insulin resistance, based on homeostatic model assessment (HOMA), were also significantly affected in subjects with the rs3741845 T allele. These findings indicate that variation in the chromosome 12 TAS2R cluster contributes to development of dysregulated postprandial glucose homeostasis in these individuals.

TABLE 6
Associations of rs2588350 with insulin and glucose
metrics in non-diabetic AFDS subjects

| | Mean trait value ± SE | | P |
|---|---|---|---|
| Trait | CC | CT/TT | value |
| Glucose Absorption | 3.16 ± 0.06 | 3.66 ± 0.15 | 0.016 |
| (mmol/l) | (n = 601) | (n = 92) | |
| Glucose 30 min | 8.17 ± 0.06 | 8.80 + 0.17 | 0.006 |
| (mmol/l) | (n = 602) | (n = 92) | |
| Glucose 60 min | 8.32 ± 0.08 | 9.12 ± 0.24 | 0.029 |
| (mmol/l) | (n = 603) | (n = 91) | |
| Glucose 90 min | 7.19 ± 0.08 | 7.84 ± 0.23 | 0.045 |
| (mmol/l) | (n = 603) | (n = 92) | |
| Glucose 120 min | 6.16 ± 0.07 | 6.63 ± 0.20 | 0.03 |
| (mmol/l) | (n = 603) | (n = 92) | |
| Insulin Response | 545.05 ± 16.28 | 683.80 + 60.18 | 0.007 |
| (pmol/l) | (n = 593) | (n = 91) | |
| Ln Insulin 30 min | 5.58 ± 0.02 | 5.74 ± 0.06 | 0.12 |
| (pmol/l) | (n = 597) | (n = 91) | |
| Ln Insulin 60 min | 5.75 ± 0.02 | 5.83 ± 0.06 | 0.54 |
| (pmol/l) | (n = 598) | (n = 91) | |
| Ln Insulin 90 min | 5.55 ± 0.03 | 5.77 ± 0.07 | 0.012 |
| (pmol/l) | (n = 598) | (n = 91) | |
| Ln Insulin 120 min | 5.23 ± 0.03 | 5.42 ± 0.08 | 0.024 |
| (pmol/l) | (n = 598) | (n = 91) | |
| Ln HOMA | 0.80 ± 0.02 | 0.87 ± 0.04 | 0.23 |
| | (n = 596) | (n = 91) | |

TABLE 7
Associations of rs3741845 with insulin and glucose
metrics in non-diabetic AFDS subjects

| | Mean trait value ± SE | | P |
|---|---|---|---|
| Trait | CC | CT/TT | value |
| Glucose Absorption | 3.13 ± 0.06 | 3.70 ± 0.12 | 0.0014 |
| (mmol/l) | (n = 539) | (n = 157) | |
| Glucose 30 min | 8.13 ± 0.07 | 8.82 ± 0.13 | 0.0006 |
| (mmol/l) | (n = 540) | (n = 157) | |
| Glucose 60 min | 8.31 ± 0.09 | 9.01 ± 0.18 | 0.012 |
| (mmol/l) | (n = 541) | (n = 156) | |
| Glucose 90 min | 7.19 ± 0.09 | 7.70 ± 0.17 | 0.054 |
| (mmol/l) | (n = 541) | (n = 157) | |
| Glucose 120 min | 6.16 ± 0.07 | 6.42 ± 0.15 | 0.311 |
| (mmol/l) | (n = 541) | (n = 157) | |
| Insulin Response | 545.35 ± 17.65 | 650.80 ± 40.33 | 0.0086 |
| (pmol/l) | (n = 532) | (n = 155) | |
| Ln Insulin 30 min | 5.58 ± 0.02 | 5.71 ± 0.05 | 0.017 |
| (pmol/l) | (n = 535) | (n = 156) | |
| Ln Insulin 60 min | 5.75 ± 0.02 | 5.85 ± 0.05 | 0.1 |
| (pmol/l) | (n = 536) | (n = 156) | |
| Ln Insulin 90 min | 5.55 ± 0.03 | 5.73 ± 0.06 | 0.0088 |
| (pmol/l) | (n = 536) | (n = 156) | |
| Ln Insulin 120 min | 5.22 ± 0.03 | 5.36 ± 0.06 | 0.046 |
| (pmol/l) | (n = 536) | (n = 156) | |
| Ln HOMA | 0.80 ± 0.02 | 0.86 ± 0.03 | 0.035 |
| | (n = 534) | (n = 156) | |

TABLE 8
Associations of rs10772420 with insulin and glucose
metrics in non-diabetic AFDS subjects

| | Mean trait value ± SE | | | P |
|---|---|---|---|---|
| Trait | AA | AG | GG | value |
| Glucose Absorption | 3.02 ± 0.08 | 3.40 ± 0.09 | 3.56 ± 0.17 | 0.003 |
| (mmol/l) | (n = 317) | (n = 259) | (n = 88) | |
| Glucose 30 min | 8.00 ± 0.09 | 8.45 ± 0.10 | 8.66 ± 0.18 | 0.002 |
| (mmol/l) | (n = 317) | (n = 259) | (n = 89) | |
| Glucose 60 min | 8.15 ± 0.12 | 8.58 ± 0.13 | 8.97 ± 0.23 | 0.017 |
| (mmol/l) | (n = 318) | (n = 258) | (n = 89) | |
| Glucose 90 min | 7.08 ± 0.11 | 7.34 ± 0.12 | 7.73 ± 0.23 | 0.03 |
| (mmol/l) | (n = 318) | (n = 259) | (n = 89) | |
| Glucose 120 min | 6.07 ± 0.10 | 6.20 ± 0.11 | 6.72 ± 0.19 | 0.016 |
| (mmol/l) | (n = 318) | (n = 259) | (n = 89) | |
| Insulin Response | 530.00 ± 20.79 | 596.32 ± 30.95 | 624.22 ± 47.82 | 0.09 |
| (pmol/l) | (n = 315) | (n = 253) | (n = 87) | |
| Ln Insulin 30 min | 5.55 ± 0.03 | 5.66 ± 0.03 | 5.67 ± 0.06 | 0.09 |
| (pmol/l) | (n = 315) | (n = 255) | (n = 89) | |
| Ln Insulin 60 min | 5.70 ± 0.03 | 5.80 ± 0.04 | 5.90 ± 0.06 | 0.04 |
| (pmol/l) | (n = 315) | (n = 256) | (n = 89) | |
| Ln insulin 90 min | 5.53 ± 0.03 | 5.62 ± 0.04 | 5.71 ± 0.06 | 0.04 |
| (pmol/l) | (n = 315) | (n = 256) | (n = 89) | |
| Ln Insulin 120 min | 5.20 ± 0.04 | 5.27 ± 0.05 | 5.39 ± 0.07 | 0.1 |
| (pmol/l) | (n = 315) | (n = 256) | (n = 89) | |
| Ln HOMA | 0.78 ± 0.02 | 0.84 ± 0.03 | 0.85 ± 0.04 | 0.5 |
| | (n = 314) | (n = 256) | (n = 88) | |

Type 2 diabetes mellitus (T2DM) is characterized by elevated plasma glucose, increased hepatic gluconeogenesis, decreased insulin mediated glucose transport and impaired beta cell function (DeFronzo et al.). The alleles associated with glucose and insulin dysregulation (rs2588350, rs3741845 and rs10772420) were also examined for their association with the presence of T2DM. The three risk allele SNPs were genotyped in a set of T2DM eases from the AFDS (n=145). Allele and genotype frequencies were then compared between T2 DM cases and a subset of normoglycemic controls (n=358) from the AFDS. Significant associations with T2DM were found for rs2588350 (P=0.0007) and rs3741845 (P 0.005), but not for rs10772420 (P=0.3). For both rs2588350 and rs3741845, the T2DM-associated allele was the same as that associated with increased glucose and insulin AUC (Table 4). As glucose dysregulation is a major risk factor for the development of T2DM, these data provide important validation of the association of rs2588350 and rs341845 with glucose and insulin homeostasis.

### Example 4

### TAS2R Function in the Gut

Because several TAS1R and TAS2R receptors are expressed in enteroendocrine cells of the gut, it is possible that TAS2R9 could impact glucose and insulin homeostasis through the regulation of incretin hormones, e.g., glucagon like peptide-1 (GLP-1), secretion from gut enteroendocrine cells. The expression of the TAS2R9 receptor in enteroendocrine cells, however, has not been disclosed to date. TAS2R9 was amplified from RNA of NCI-H716 cells, a hum an enteroendocrine L cell line, by reverse transcription-polymerase chain reaction. Briefly, total RN A was isolated from human enteroendocrine NCI-H716 cells with Trizol reagent, then reverse transcribed with random hexamer probes. A reaction without reverse transcriptase was included to control for genomic DNA contamination. TAS2R9 (GeneID: 50835) cDNA was amplified using gene specific primers within the single coding exon. TAS1R3 (GeneID: 83756) cDNA was amplified using gene specific primers exons 4 and 6. These same primer pairs were used to amplify TAS2R9 and TAS1R3 products from human cDNA (Biochain Institute, Hayward, Calif.). All PCR products were verified by sequencing.

A TAS2R9 product was also amplified from human cecum RNA by RT-PCR. The TAS2R9 products were amplified from cDNA and not genomic DNA contaminants: PCR from control samples that were not reverse transcribed gave no TAS2R9 product (not shown), and oligos that recognize coding sequences in exons 4 and 6 of taste receptor TAS1R3 amplify a product lacking the two intervening introns.

### Example 5

### Identification of Novel SNPs

Novel SNPs associated with a trait of an obesity disorder or an obesity disorder are identified by amplifying TAS1R and TAS2R genes or gene fragments, as well as adjacent genomic DNA, by PCR from individuals who are affected or are unaffected by traits of an obesity disorder or an obesity disorder. DNA sequencing of the PCR amplicons is then performed before or after subcloning into bacterial plasmids or other DNA vectors. “Novel SNPs” is used herein to mean SNPs that have not been reported in the literature or are not publicly (e.g., dbSNP database, International HapMap Project database, or UCSC Genome Browser or commercially available. Novel SNPs are genotyped using the Applied Biosystem's Taqman platform in additional individuals affected or unaffected by traits of an obesity disorder or an obesity disorder.

The novel SNPs are then tested for association with discrete or continuous traits associated with a trait of an obesity disorder or an obesity disorder by determining an association between a particular genotype and a phenotype correlated with a trait of an obesity disorder or an obesity disorder, e.g., via parametric or non-parametric analysis.

### LIST OF REFERENCES INCORPORATED BY REFERENCE

Bartoshuk L M, Duffy V B, Hayes J E, Moskowitz H R, Snyder D J. (2006) Psychophysics of sweet and fat perception in obesity: problems, solutions and new perspectives. Philos Trans R Soc Lond B Biol Sci., 361(1471), 1137-48.
Batoshuk, L. M. (2006) Genetic and pathological variation in the perception of sweetness. ACS Symposium on Sweetness and Sweeteners. In press.
Bezencon C, le Coutre J, Damak S. (2007) Taste-signaling proteins are coexpressed in solitary intestinal epithelial cells. Chem Senses., 32(1), 41-9, Epub 2006 Oct. 9.
Drewnowski, A., Henderson, S. A., Shore, A. B. and Barratt-Fornell, A. (1997) Nontasters, tasters, and supertasters of 6-n-propylthiouracil (PROP) and hedonic response to sweet. Physiol Behav, 62, 649-655.
Duffy V B. (2004) Associations between oral sensation, dietary behaviors and risk of cardiovascular disease (CVD). Appetite, 43(1), 5-9.
Duffy, V. B. and Bartoshuk, L. M. (2000) Food acceptance and genetic variation in taste. J Am Diet Assoc, 100, 647-655.
Dyer, J., Salmon, K. S., Zibrik, L. and Shirazi-Beechey, S. P. (2005) Expression of sweet taste receptors of the T1 R family in the intestinal tract and enteroendocrine cells. Biochem Soc Trans, 33, 302-305.
Enoch, M. A., Harris, C. R. and Goldman, D. (2001) Does a reduced sensitivity to bitter taste increase the risk of becoming nicotine addicted? Addict Behav 26, 399-404.
Goldstein G L, Daun H, Tepper B J. (2005) Adiposity in middle-aged women is associated with genetic taste blindness to 6-n-propylthiouracil. Obes Res., 13(6), 1017-23.
Hinrichs A L, Wang J C, Bufe B, Kwon J M, Budde J, Allen R, Bertelsen S, Evans W, Dick D, Rice J, Foroud T. Nurnberger J, Tischfield J A, Kupermnan S, Crowe R, Hesselbrock V, Schuckit M, Almasy L, Porjesz B, Edenberg H J, Begleiter H, Meyerhof W, Bierut L J, Goate A M. (2206) Functional variant in a bitter-taste receptor (hTAS2R16) influences risk of alcohol dependence. Am J Hum Genet, 78, 103-11
Kim, U., Wooding, S., Ricci, D., Jorde, L. B. and Drayna, D. (2005) Worldwide haplotype diversity and coding sequence variation at human bitter taste receptor loci. Hum Mutat, 26, 199-204.
Kim, U. K., Breslin, P. A., Reed, D. and Drayna, D. (2004) Genetics of human taste perception. J Dent Res, 83, 448-453.
Kim U. K., Jorgenson, E., Coon, H., Leppert, M., Risch, N. and Drayna, D. (2003) Positional cloning of the human quantitative trait locus underlying taste sensitivity to phenylthiocarbamide. Science, 299, 1221-1225.
Lin, H. F., Juo, S. H. and Cheng, R. (2005) Comparison of the power between microsatellite and single-nucleotide polymorphism markers for linkage and linkage disequilibrium mapping of an electrophysiological phenotype. BMC Genet, 6 Suppl 1, S7.
Lugaz, O., Pillias, A. M. and Faurion, A. (2002) A new specific ageusia: some humans cannot taste L-glutamate. Chem Senses, 27, 105-115.
Mennella, J. A., Pepino, M. Y. and Reed, D. R. (2005) Genetic and environmental determinants of bitter perception and sweet preferences. Pediatrics, 115, e216-222.
Nelson, T. M., Munger, S. D. and Boughter, J. D. (2005) Haplotypes at the Tas2r locus on distal chromosome 6 vary with quinine taste sensitivity in inbred mice. BMC Genetics, 6, 32.
Nie, Y., Hobbs, J. R., Viges, S., Olson, W. J., Conn, G. L. and Munger, S. D. (2006) Expression and purification of functional ligand-binding domains of T1R3 taste receptors. Chem Senses, In press,
Nie, Y., Vigues, S., Hobbs, J. R., Conn, G C. L. and Munger, S. D. (2005) Distinct contributions of T1R2 and T1R3 taste receptor subunits to the detection of sweet stimuli. Curr Biol, 15, 1948-1952.
Scott, K. (2005) Taste recognition, food for thought. Neuron, 48, 455-464.
Tepper, B. J. and Nurse, R. J. (1997) Fat perception is related to PROP taster status. Physiol Behav, 61, 949-954.
Tepper, B. J. and Nurse, R. J. (1998) PROP taster status is related to fat perception and preference. Ann N Y Acad Sci, 855, 802-804.
Tepper B. J., Ullrich N. V. (2002) Influence of genetic taste sensitivity to 6-n-propylthiouracil (PROP), dietary restraint and disinhibition on body mass index in middle-aged women. Physiol Behav., 75(3), 305-12.
Timpson, N. J., Christensen, M., Lawlor, D. A., Gaunt, T. R., Day, I. N., Ebrahim, S. and Davey Smith, G. (2005) TAS2R38 (phenylthiocarbamide) haplotypes, coronary heart disease traits, and eating behavior in the British Women's Heart and Health Study. Am J Clin Nutr, 1005-1011.
Wang J. C., Hinrichs A. L., Bertelsen S., Stock H., Budde J. P., Dick D. M., Bucholz K. K., Rice J., Saccone N., Edenberg H. J., Hesselbrock V., Kuperman S., Schuckit M. A. Bierut L. J., Goate A. M. (2007) Functional variants in TAS2R38 and TAS2R16 influence alcohol consumption in high-risk families of African-American origin. Alcohol Clin Exp Res., 31(2), 209-15.
Wu, S. V., Rozengurt, N., Yang, M., Young, S. H., Sinnett-Smith, J. and Rozengurt, E. (2002) Expression of bitter taste receptors of the T2R family in the gastrointestinal tract and enteroendocrine STC-1 cells. Proc Natl Acad Sci USA, 99, 2392-2397.
Jang, H. J., Kokrashvili, Z., Theodorakis, M. J., Carlson, O. D., Kim, B. J., Zhou, J., Kim, H. H., Xu, X., Chan, S. L., Juhaszova, M., et al. 2007. Gut-expressed gustducin and taste receptors regulate secretion of glucagon-like peptide-1. Proc Natl Acad Sci USA.
Pronin, A. N., Xu, H., Tang, H., Zhang, L., Li, Q., and Li, X. 2007. Specific Alleles of Bitter Receptor Genes Influence Human Sensitivity to the Bitterness of Aloin and Saccharin. Curr Biol.
Hseuh, W. C., Mitchell, B. D., Aburomia, R., Pollin, T., Sakul, T., Gelder Ehm, M., Michelsen, U. K., Wagner, M. J., St Jean, P. L., Knowler, W. C., et al. 2000. Diabetes in the Old Order Amish: characterization and heritability analysis of the Amish Family Diabetes Study. Diabetes Care 23:595-601.
The International HapMap Consortium. 2005. A haplotype map of the human genome. Nature 437.1299-1320.
Sherry, S. T., Ward, M. H., Kholodov, M., Baker, J., Plan, L., Smigielski, E. M., and Sirotkin, K. 2001. dbSNP: the NCBI database of genetic variation. Nucleic Acids Res 29:308-311.
DeFronzo, R. A. 2004. Pathogenesis of type 2 diabetes mellitus. Med Clin North Am 88:787-835, ix.
Barrett, J. C., Fry, B., Maller, J., and Daly, M. J. 2005. Haploview: analysis and visualization of LD and haplotype maps. Bioinformatics 21:263-265.
Gabriel. S. B., Schaffner, S. F., Nguyen, H., Moore, J. M., Roy, J., Blumenstiel, B., Higgins, J., DeFelice, M., Lochner, A., Faggart, M., et al. 2002. The structure of haplotype blocks in the human genome. Science 296:2225-2229.
Bufe, B., Hofmann, T., Krautwurst, D., Raguse, J. D., and Meyerhof, W. 2002. The human TAS2R16 receptor mediates bitter taste in response to beta-glucopyranosides. Nat Genet 32:397-401.
Kim, U. K., Wooding, S., Riaz, N., Jorde, L. B., and Drayna, D. (2006). Variation in the human TAS1R taste receptor genes. Chem Senses 31, 599-611.
Margoiskee, R. F., Dyer, J., Kokrashvili, Z., Salmon, K. S., Ilegems, E., Daly, K., Maillet, E. L., Ninomiya, Y., Mosinger, B., and Shirazi-Beechey, S. P. (2007). TIR3 and gustducin in gut sense sugars to regulate expression of Na+-glucose cotransporter 1. Proc Natl Acad Sci USA.
Jang, H. J., Kokrashvili, Z., Theodorakis, M. J., Carlson, O. D., Kim, B. J., Zhou, J., Kim, H. H., Xu, X., Chan, S. L., Juhaszova, M., Bernier, M., Mosinger, B., Margolskee, R. F., and Egan, J. M. (2007). Gut-expressed gustducin and taste receptors regulate secretion of glucagon-like peptide-1. Proc Natl Acad Sci USA.
Mace, O. J., Affleck, J., Patel, N., and Kellett, G. L. (2007). Sweet taste receptors in rat small intestine stimulate glucose absorption through apical GLUT2. J Physiol 582, 379-392.
Ueda, T., Ugawa, S., Yamamura, H., Imaizumi, Y., and Shimada, S. 2003. Functional interaction between T2R taste receptors and G-protein alpha subunits expressed in taste receptor cells. J Neurosci 23:7376-7380.
Sternini, C., Anselmi, L., and Rozengurt, F. (2008). Enteroendocrine cells: a site of ‘taste’ in gastrointestinal chemosensing. Curr Opin Endocrinol Diabetes Obes 15, 73-78.

What is claimed is:

1. A method of determining an increased risk of a subject to acquire a trait of an obesity disorder, the method comprising
a) determining a test genetic sequence of a genetic locus comprising at least one portion of at least one taste receptor gene in the subject; and
b) reviewing the test genetic sequence(s) for the presence of at least one risk allele associated with a taste receptor,
wherein the presence of at least one risk allele associated with a taste receptor indicates an increased risk of the subject for acquiring an obesity disorder or a trait of an obesity disorder.

2. The method of claim 1, wherein the at least one taste receptor gene is a TAS1R gene or a TAS2R gene.

3. The method of claim 2, wherein the test genetic sequence comprises a single nucleotide polyrmorphismn (SNP) associated a TAS1R gene or a TAS2R gene.

4. The method of claim 3, wherein the at least one taste receptor gene is a TAS1R gene.

5. The method of claim 3, wherein the at least one taste receptor gene is a TAS2R gene.

6. The method of claim 4, wherein the at least one TAS1R gene is selected from the group consisting of TAS1R1. TAS1R2 and TAS1R3.

7. The method of claim 5, wherein the at least one TAS2R gene is selected from the group consisting of TAS2R9, TAS2R39, TAS2R40, TAS2R41, TAS2R42, TAS2R48 and TAS2R60.

8. The method of claim 3, wherein the at least one SNP comprises the rs3741845 SNP.

9. The method of claim 3, wherein the at least one SNP comprises the rs4726600 SNP.

10. The method of claim 3, wherein the at least one SNP comprises the rs5020531 SNP.

11. The method of claim 3, wherein the at least one SNP comprises the rs4595035 SNP.

12. The method of claim 3, wherein the at least one SNP comprises the rs10278721 SNP.

13. The method of claim 3, wherein the at least one SNP comprises the rs534126 SNP.

14. The method of claim 3, wherein the at least one SNP comprises the rs10241042 SNP.

15. The method of claim 3, wherein the at least one SNP comprises the rs12036097 SNP.

16. The method of claim 3, wherein the at least one SNP comprises the rs12567264 SNP.

17. The method of claim 3, wherein the at least one SNP comprises the rs12408808 SNP.

18. The method of claim 3, wherein the at least one SNP comprises the rs10772420 SNP.

19. The method of claim 3, wherein the at least one SNP comprises the rs25883580 SNP.

20. The method of claim 1, wherein the trait of the obesity disorder is selected from the group consisting of high total cholesterol, low high-density lipoprotein (HDL) cholesterol, impaired fasting glucose levels, hyperproinsuliinemia, thyroid dysfunction, increased body-mass index (BMI), hypertension, obesity, impaired glucose tolerance levels, metabolic syndrome and type 2 diabetes, eating behavior, and lifespan.

21. The method of claim 20, wherein the trait of the obesity disorder is impaired glucose tolerance levels.

22. The method of claim 20, wherein the trait of the obesity disorder is type 2 diabetes.

23. The method of claim 20, wherein the trait of the obesity disorder is metabolic syndrome.

24. A method of determining a novel risk allele associated with a trait of an obesity disorder or an obesity disorder, the method comprising:
a) genotyping at least one test genetic sequence of a genetic locus, said locus comprising at least one portion of at least one taste receptor gene from individuals who possess a known risk allele associated with a trait of an obesity disorder or an obesity disorder; and
b) comparing the test genetic sequence to the genetic sequence of a control individual to determine a difference between the test genetic sequence and a control genetic sequence, and
c) comparing said differences to known alleles to determine the identity of a novel risk allele,
said risk allele being associated with an obesity related disorder or a trait of an obesity related disorder.

25. A method of altering the levels of incretin hormones secreted from enteroendocrine cells, the method comprising administering to the cells a compound that affects the activity of a TAS2R9 taste receptor present on the surface of said enteroendocrine cells, wherein affecting the activity of the TAS2R9 taste receptor will alter the secretion of incretin hormones from the enteroendocrine cells.

26. The method of claim 25, wherein the levels of incretin hormones secreted from the enteroendocrine cells are reduced by administering a compound that reduces the activity of the TAS2R9 receptor.

27. The method of claim 26, wherein the incretin hormone is glucagon like protein 1 (GLP-1).

28. The method of claim 26, wherein the TAS2R9 receptor is a wild-type receptor or a mutant receptor.
